(19) United States
(12) Patent Application Publication
(10) Pub. No.: US 2022/0392625 A1
(43) Pub. Date: Dec. 8, 2022

(54) METHOD AND SYSTEM FOR AN INTERFACE TO PROVIDE ACTIVITY RECOMMENDATIONS

(71) Applicant: LULULEMON ATHLETICA CANADA INC., Vancouver (CA)

(72) Inventors: Sian Victoria ALLEN, Vancouver (CA); Thomas McCarthy WALLER, Vancouver (CA); Peder Richard Douglas SANDE, Vancouver (CA); Amanda Susanne CASGAR, Vancouver (CA); Robert John GATHERCOLE, Vancouver (CA)

(21) Appl. No.: 17/772,663

(22) PCT Filed: Oct. 29, 2020
(86) PCT No.: PCT/CA2020/051454

§ 371 Date: Apr. 28, 2022

Related U.S. Application Data
(60) Provisional application No. 62/928,210, filed on Oct. 30, 2019.
(60) Provisional application No. 63/052,836, filed on Jul. 16, 2020.

(51) Int. Cl. G16H 40/63; G16H 20/30

(52) U.S. Cl. CPC G16H 40/63; G16H 20/30

(57) ABSTRACT

There is described a system for providing an interface with activity recommendations by monitoring user activity in which user data relating to a user is received at a user device. The user data comprises at least image data, text input, biometric data, and audio data, and may have been captured using one or more sensors on the user device. The user data is processed using at least: facial analysis; body analysis; eye tracking; voice analysis; behavioural analysis; social network analysis; location analysis; user's activities analysis; and text analysis. Based on the user data, one or more states of one or more cognitive-affective competencies of the user may be determined. An emotional signature of the user is determined, based on the one or more states of the one or more cognitive-affective competencies of the user. Based on the emotional signature, one or more recommendations for improving the emotional signature may be recommended.

### CROSS-REFERENCE TO RELATED APPLICATIONS

The present disclosure claims the benefit of and priority to U.S. Provisional Patent Applications Nos. 62/928,210 filed Oct. 30, 2019 and 63/052,836 filed Jul. 16, 2020, the contents of which are hereby incorporated herein by reference.

### FIELD

The present disclosure relates to methods and systems for an interface to provide activity recommendations by monitoring user activity with sensors, and methods and systems for determining an emotional signature of a user, and to generating the activity recommendations based on the emotional signature of the user for improving user's wellbeing.

### BACKGROUND

Human emotions are highly complex and can vary significantly from one moment to the next. While a person's personality type may remain relatively fixed over long time frames, a person's mood (e.g. whether they are currently feeling happy, sad, or angry) may vary much more rapidly, depending on the particular environment or social situation in which the person finds themselves. In addition, the levels or states of a person's cognitive-affective competencies (e.g. how they process external and internal stimuli leading to biases in attention regulation, emotion regulation, prosociality, and non-attachment) may also vary over relatively short time frames, depending for example on the individual's particular physical and mental condition (e.g. the amount of sleep they have had, or their current level of hunger).

Embodiments described herein relate to automated systems for detecting a person's personality type, mood, and other emotional characteristics through the use of invasive and non-invasive sensors. As such, it is possible to attempt to establish a person's current emotional state based on, for example, data for their facial expressions or the tone of their voice as captured by various different sensors.

A person who is exhibiting a relatively poor state of emotional wellbeing may be in need of psychological or emotional assistance, and there exist many different types of activities, coaching sessions, and therapies that may be used to assist the person in boosting their general emotional fitness or wellbeing. Embodiments described herein involve automated systems for providing activity recommendations with assistance tailored to an individual's specific personality and current state of emotional wellbeing as captured by sensors.

### SUMMARY

Embodiments relate to methods and systems with non-transitory memory storing data records for user data across multiple channels, such as image data relating to the user, text input relating to the user, data defining physical or behavioural characteristics of the user, and audio data relating to the user; and a hardware processor having an interface to provide activity recommendations generated based on the user data and activity metrics, and the hardware processor can access the user data stored in the memory to determine an emotional signature of a user, and generate the activity recommendations by accessing a non-transitory memory storing a set of activity records located based on the emotional signature of the user and ranked for improving user's wellbeing.

Embodiments relate to a system for monitoring a user over a user session using one or more sensors and providing an interface with activity recommendations for the user session. The system has non-transitory memory storing activity recommendation records, emotional signature records, and user records storing user data received from a plurality of channels, wherein the user data comprises image data relating to the user, text input relating to the user, data defining physical or behavioural characteristics of the user, and audio data relating to the user. The system has a hardware processor programmed with executable instructions for an interface for obtaining user data for a user session over a time period, transmitting a recommendation request for the user session, and providing activity recommendations for the user session received in response to the recommendation request. The system has a hardware server coupled to the memory to access the activity recommendation records, the emotional signature records, and the user records. The hardware server is programmed with executable instructions to transmit the activity recommendations to the interface over a network in response to receiving the recommendation request from the interface by: computing activity metrics, cognitive-affective competency metrics, and social metrics using the user data for the user session and the user records by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis; computing one or more states of one or more cognitive-affective competencies of the user based on the cognitive-affective competency metrics and the social metrics; computing an emotional signature of the user based on the one or more states of the one or more cognitive-affective competencies of the user and using the emotional signature records; and computing the activity recommendations based on the emotional signature of the user, the activity metrics, the activity recommendation records, and the user records. The system has a user device comprising one or more sensors for capturing user data during the time period, and a transmitter for transmitting the captured user data to the interface or the hardware server over the network to compute the activity recommendations.

In some embodiments, the hardware server computes activity metrics, cognitive-affective competency metrics, and social metrics with classifiers using the user data for the user session and the user records and multimodal feature extraction that: for the image data and the data defining the physical or behavioural characteristics of the user, implements at least one of: facial analysis; body analysis; eye tracking; behavioural analysis; social network or graph analysis; location analysis; user activity analysis; for the audio data, implements voice analysis; and for the text input implements text analysis; In some embodiments, the non-transitory memory stores classifiers for generating data defining physical or behavioural characteristics of the user, and the hardware server computes the activity metrics, cognitive-affective competency metrics, and social metrics using the classifiers and features extracted from the multimodal feature extraction.

In some embodiments, the non-transitory memory stores a user model corresponding to the user and the hardware server computes the emotional signature of the user using the user model.

In some embodiments, the user device connects to or integrates with an immersive hardware device that captures the audio data, the image data and the data defining the physical or behavioural characteristics of the user.

In some embodiments, the non-transitory memory has a content repository and the hardware server has a content curation engine that maps the activity recommendations to recommended content and transmits the recommended content to the interface.

In some embodiments, the hardware processor programmed with executable instructions for the interface further comprises a voice interface for communicating activity recommendations for the user session received in response to the recommendation request.

In some embodiments, the hardware processor couples to a memory storing mood classifiers to capture the data defining physical or behavioural characteristics of the user.

In some embodiments, the system has one or more modulators in communication with one or more ambient fixtures to change external sensory environment based on the activity recommendations, the one or more modulators being in communication with the hardware server to automatically modulate the external sensory environment of the user during the user session.

In some embodiments, the one or more ambient fixtures comprise at least one of a lightening fixture, an audio system, an aroma diffuser, a temperature regulating system.

In some embodiments, the system has a plurality of user devices, each having different types of sensors for capturing different types of user data during the user session, each of the plurality of devices transmitting the captured different types of user data to the hardware server over the network to compute the activity recommendations.

In some embodiments, the system has a plurality of hardware processors for a group of users, each hardware processor programmed with executable instructions for a corresponding interface for obtaining user data for a corresponding user of the group of users for the user session over the time period, and providing activity recommendations for the user session received in response to the recommendation request, wherein the hardware server transmits the activity recommendations to the corresponding interfaces of the plurality of hardware processors in response to receiving the recommendation request from the corresponding interfaces and computes the activity recommendations for the group of users.

In some embodiments, the hardware server is configured to determine an emotional signature of one or more additional users; determine users with similar emotional signatures; predict connectedness between users with similar emotional signatures; and generate the activity recommendations for the users with similar emotional signatures.

In some embodiments, the interface can receive feedback on the activity recommendations for the user session, transmit the feedback to the hardware server.

In some embodiments, the interface can transmit another recommendation request for the user session, and provide additional activity recommendations for the user session received in response to the other recommendation request.

In some embodiments, the interface obtains additional user data after providing the activity recommendations for the user session, the additional user data captured during performance of the activity recommendations by the user.

In some embodiments, the interface transmits another recommendation request for another user session, and provides updated activity recommendations for the other user session received in response to the other recommendation request, the updated activity recommendations being different that the activity recommendations.

In some embodiments, the one or more activity recommendations comprise a pre-determined content for display or playback on the hardware processor.

In some embodiments, the interface is a coaching application and the one or more recommended activity is delivered by a matching coach.

In some embodiments, the activity recommendations are pre-determined classes selected from a set of classes stored in the activity recommendation records.

In some embodiments, the activity recommendations are a program with variety of content for the interface to guide user's interactions or experience for a prolong time.

Embodiments relate to a computer-implemented method. The method involves receiving user data relating to a user from a plurality of channels at a hardware server and storing the user data as user records in non-transitory memory, wherein the user data comprises image data relating to the user, text input relating to the user, data defining physical or behavioural characteristics of the user, and audio data relating to the user; generating activity metrics, cognitive-affective competency metrics, and social metrics by processing the user data using one or more hardware processors configured to process the user data from the plurality of channels by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis; determining, based on the cognitive-affective competency metrics and social metrics generated from the processed user data, one or more states of one or more cognitive-affective competencies of the user; determining an emotional signature of the user based on the one or more states of the one or more cognitive-affective competencies of the user; and automatically generating, based on the emotional signature of the user and the activity metrics, one or more activity recommendations for a user session; transmitting the activity recommendations to a user interface at a hardware processor in response to a recommendation request; updating the user interface at the hardware processor to provide the activity recommendations based on user preferences; and modulating an external sensory actuators of an external sensory environment during the recommended activity in response to the hardware server or interface.

In some embodiments, the one or more activity recommendations comprise a pre-determined content.

In some embodiments, the one or more activity recommendation is delivered by a matching coach.

In some embodiments, the one or more activity recommendations are pre-determined classes.

In some embodiments, the one or more activity recommendations are a program with variety of content to guide user's interactions or experience for a prolong time.

In some embodiments, the program comprises two or more phases, each phase having a different content, intensity or duration.

In some embodiments, the modulating of the external sensory actuators comprises modulating at least one of a sound, lighting, smell, temperature or an air flow device.

In some embodiments, the method further involves receiving user data relating to one or more additional users, wherein the user data comprises at least one of image data relating to the one or more additional users, text input relating to the one or more additional users, biometric data relating to the one or more additional users, and audio data relating to the one or more additional users; processing the user data using at least one of: facial analysis; body analysis; eye tracking; voice analysis; behavioural analysis; social network analysis; location analysis; user activity analysis; and text analysis; determining, based on the processed user data, one or more states of one or more cognitive-affective competencies of the one or more additional users; determining an emotional signature of each of the one or more additional users; determining users with similar emotional signatures; predicting connectedness between users with similar emotional signatures; and generating one or more activity recommendations for transmission to interfaces of users with similar emotional signatures.

In some embodiments, the method further involves determining, based on the processed user data, a personality type of the user, wherein determining the emotional signature of the user is further based on the personality type of the user.

In some embodiments, the processed user data comprises personality type data, and wherein determining the personality type of the user comprises: comparing the personality type data to stored personality type data indicative of correlations between personality types and personality type data.

In some embodiments, the processed user data comprises cognitive-affective competency data, and wherein determining the one or more states of the one or more cognitive-affective competencies of the user comprises: comparing the cognitive-affective competency data to stored cognitive-affective competency data indicative of correlations between states of cognitive-affective competencies and cognitive-affective competency data.

In some embodiments, the method further involves determining, based on the processed user data, at least one of: one or more mood states of the user, one or more attentional states of the user, one or more prosociality states of the user, one or more motivational states of the user, one or more reappraisal states of the user, and one or more insight states of the user, and wherein determining the one or more states of the one or more cognitive-affective competencies of the user is further based on the at least one of: the one or more mood states of the user, the one or more attentional states of the user, the one or more prosociality states of the user, the one or more motivational states of the user, the one or more reappraisal states of the user, and the one or more insight states of the user.

Embodiments relate to a system with one or more hardware servers with non-transitory memory storing associations between emotional signatures and recommendations; a network; and a user device comprising one or more sensors and being operable to communicate with the one or more servers over the network, wherein the user device has a hardware processor that is programmed with machine executable instructions to: use the one or more sensors to receive user data relating to a user during a time period for a user session, wherein the user data comprises image data relating to the user, text input relating to the user, data defining physical or behavioural characteristics of the user, and audio data relating to the user; and transmit over the network the user data to the one or more servers, and wherein the one or more servers are configured to: generate activity metrics, cognitive-affective competency metrics, and social metrics using one or more processors configured to process the user data from the one or more sensors by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis; determine, based on the cognitive-affective competency metrics and social metrics generated from the processed user data, one or more states of one or more cognitive-affective competencies of the user; determine an emotional signature of the user based on the one or more states of the one or more cognitive-affective competencies of the user; and automatically generate one or more, based on the emotional signature of the user and the activity metrics, activity recommendations and transmit the activity recommendations an interface of the user device in response to a recommendation request.

In some embodiments, the one or more activity recommendations comprise a pre-determined content.

In some embodiments, the one or more activity recommendations are delivered by a matching coach.

In some embodiments, the one or more recommended activity is a pre-determined class.

In some embodiments, wherein the one or more recommended activity is a program with variety of content to guide user's interactions or experience for a prolong time.

In some embodiments, the system has one or more modulators in communication with one or more ambient fixtures to change to change external sensory environment, the one or more modulators being in communication with the one or more servers to automatically modulate the external sensory environment of the user during the recommended activity.

In some embodiments, the one or more ambient fixtures comprise at least one of a lightening fixture, an audio system, an aroma diffuser, a temperature regulating system.

In some embodiments, the one or more servers are configured to determine an emotional signature of one or more additional users; determine users with similar emotional signatures; predict connectedness between users with similar emotional signatures; and generate one or more activity recommendations to users with similar emotional signatures.

According to an aspect of the disclosure, there is provided a computer-implemented method comprising: receiving user data relating to a user from a plurality of channels, wherein the user data comprises image data relating to the user, text input relating to the user, data defining physical or behavioural characteristics of the user, and audio data relating to the user; generating activity metrics, cognitive-affective competency metrics, and social metrics by processing the user data using one or more processors configured to process the user data from the plurality of channels by: for the image data and the data defining the physical or behavioural characteristics of the user, using at least one of: facial analysis; body analysis; eye tracking; behavioural analysis; social network or graph analysis; location analysis; user activity analysis; or the audio data, using voice analysis; and for the text input using text analysis; determining, based on the cognitive-affective competency metrics and social metrics generated from the processed user data, one or more states of one or more cognitive-affective competencies of the user; determining an emotional signature of the user based on the one or more states of the one or more cognitive-affective competencies of the user; and automatically generating, based on the emotional signature of the user and the activity metrics, one or more activity recommendations and transmitting the activity recommendations to a user interface in response to a recommendation request.

The method may further comprise, receiving user data relating to one or more additional users, wherein the user data comprises at least one of image data relating to the one or more additional users, text input relating to the one or more additional users, biometric data relating to the one or more additional users, and audio data relating to the one or more additional users; processing the user data using at least one of: facial analysis; body analysis; eye tracking; voice analysis; behavioural analysis; social network analysis; location analysis; user activity analysis; and text analysis; determining, based on the processed user data, one or more states of one or more cognitive-affective competencies of the one or more additional users; determining an emotional signature of each of the one or more additional users; determining users with similar emotional signatures; predicting connectedness between users with similar emotional signatures; and generating one or more activity recommendations to users with similar emotional signatures.

The method may further comprise determining, based on the processed user data, a personality type of the user. Determining the emotional signature of the user may be further based on the personality type of the user.

The processed user data may comprise personality type data, and determining the personality type of the user may comprise: comparing the personality type data to stored personality type data indicative of correlations between personality types and personality type data.

The method may further comprise modulating an external sensory environment to alter user's interoceptive ability to deliver greater physiological and psychological benefits during the recommended activity.

According to a further aspect of the disclosure, there is provided a system comprising: one or more servers storing associations between emotional signatures and recommendations; a network; and a user device comprising one or more sensors and being operable to communicate with the one or more servers over the network, wherein the user device is configured to: use the one or more sensors to receive user data relating to a user, wherein the user data image data relating to the user, text input relating to the user, data defining physical or behavioural characteristics of the user, and audio data relating to the user; and transmit over the network the user data to the one or more servers, and wherein the one or more servers are configured to: process the user data and generate activity metrics, cognitive-affective competency metrics, and social metrics using one or more processors configured to process the user data from the one or more sensors by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis; determine, based on the cognitive-affective competency metrics and social metrics generated from the processed user data, one or more states of one or more cognitive-affective competencies of the user; determine an emotional signature of the user based on the one or more states of the one or more cognitive-affective competencies of the user; and automatically generate, based on the emotional signature of the user and the activity metrics, activity recommendations and transmitting the activity recommendations to the user device in response to a recommendation request.

This summary does not necessarily describe the entire scope of all aspects. Other aspects, features and advantages will be apparent to those of ordinary skill in the art upon review of the following description of specific embodiments.

### BRIEF DESCRIPTION OF THE DRAWINGS

Embodiments of the disclosure will now be described in conjunction with the accompanying drawings of which:

FIG. 1 shows a system for generating recommendations for users based on their emotional signatures, according to embodiments of the disclosure;

FIG. 2 shows a user device that may be used by users of the recommendation system of FIG. 1, according to embodiments of the disclosure;

FIG. 3 shows an example relationship between user data, cognitive-affective state detection types, cognitive-affective competencies, and personality type, according to embodiments of the disclosure;

FIG. 4 shows a plot of emotional fitness as a function of time, according to embodiments of the disclosure;

FIG. 5 shows a flow diagram of a method for generating recommendations for users based on their emotional signatures, according to embodiments of the disclosure;

FIGS. 6 and 7 show examples of users improving their emotional wellbeing through interaction with the recommendation system described herein;

FIG. 8 shows a diagram of an example of components of a wellbeing platform employing a recommendation system according to embodiments of the disclosure;

FIG. 9 shows a diagram of an example computing device;

FIG. 10 shows an example system for an interface that provides activity recommendations; and

FIG. 11 shows another example system for an interface that provides activity recommendations.

FIG. 12 shows another example system for an interface that provides activity recommendations.

FIG. [symbol] shows an example user interface that provides activity recommendations.

FIG. 14 shows another example interface that provides activity recommendations.

### DETAILED DESCRIPTION

Embodiments relate to methods and systems with non-transitory memory storing data records for user data across multiple channels, such as image data relating to the user, text input relating to the user, data defining physical or behavioural characteristics of the user, and audio data relating to the user; and a hardware processor having an interface to provide activity recommendations generated based on the user data and activity metrics, and the hardware processor can access the user data stored in the memory to determine an emotional signature of a user, and generate the activity recommendations by accessing a non-transitory memory storing a set of activity records located based on the emotional signature of the user and ranked for improving user's wellbeing. The interface can display visual elements generated from the set of activity records located based on the emotional signature of the user, or otherwise communicate activity recommendations, such as via audio data or video data. The display of the visual elements can be controlled by the hardware processor based on the emotional signature of the user and ranked activities.

The present disclosure seeks to provide improved methods and systems for generating recommendations for users based on their emotional signatures. While various embodiments of the disclosure are described below, the disclosure is not limited to these embodiments, and variations of these embodiments may well fall within the scope of the disclosure which is to be limited only by the appended claims.

Generally, according to embodiments of the disclosure, there are described methods and systems for determining the emotional signature of a user. The emotional signature may be a composite metric derived from the combination of a measure of a personality type of the user (e.g. a measure of, for example, the user's openness/intellect, conscientiousness, extraversion, agreeableness, and neuroticism/emotional stability) and levels or states of cognitive-affective processes or competencies (e.g. attention, emotion regulation, awareness, compassion, etc.).

In order to establish the emotional signature, devices described herein may use one or more sensors to capture user data relating to the user. The sensors may include, for example, audio sensors (such as a microphone), optical sensors (such as a camera), tactile sensors (such as a user interface), biometric sensors (such as a heart monitor, blood pressure monitor, skin wetness monitor, electroencephalogram (EEG) electrode, etc.), location/position sensors (such as GPS) and motion detection or motion capturing sensors (such as accelerometers) for obtaining the user data. The user data may then be processed (using, for example, any of various face and body modelling or analysis techniques) and compared to stored, reference user data to determine the user's personality type and states of cognitive-affective competencies. For example, the processed user data may be used to determine one or more of the user's current mood states which in turn may assist in determining the user's personality type and states of cognitive-affective competencies.

In addition, by monitoring the individual's emotional signature over time, the methods and systems described herein may determine whether the emotional signature is improving or deteriorating. The individual's “baseline” emotional signature may be calculated over time, for example by collecting averages on the individual's states or levels of cognitive-affective competencies. Through repeated interventions over time, the levels of these competencies may increase. Thus, a user's baseline emotional signature may improve over time. The baseline emotional signature may comprise the levels or states of the user's cognitive-affective competencies, in combination with the user's personality type, averaged over a period of time.

After determining the user's emotional signature, one or more recommendations for improving the emotional signature may be generated. The recommendations may be based on recommendations that have shown, in connection with similar emotional signatures of other users, to show an improvement in the emotional signature in response to the recommendations being carried out. Depending on the evolution of the user's emotional signature over time, the recommendations may be adjusted. For example, a recommendation that has proven, once carried out by a user, to lead to an improvement in that user's emotional signature, may also be generated for a different user that is exhibiting a similar emotional signature.

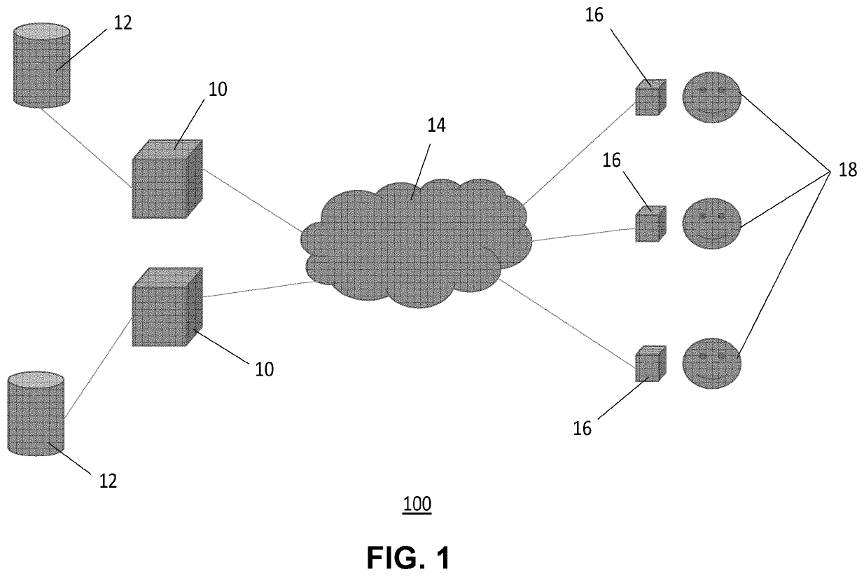

Turning to FIG. 1, there is shown an embodiment of a recommendation system 100 that may implement the methods described herein. Recommendation system 100 comprises hardware servers 10, databases 12 stored on non-transitory memory, a network 14, and user devices 16. Servers 10 have hardware processors that are communicatively coupled to databases 12 stored on the non-transitory memory, and are operable to access data stored on databases 12. Servers 10 are further communicatively coupled to user devices 16 via network 14 (such as the Internet). Thus, data may be transferred between servers 12 and user devices 16 by transmitting the data using network 14. The servers 10 include non-transitory computer readable storage medium storing instructions to configure one or more hardware processors to provide an interface for collecting sensor data, and exchanging data and commands with other components of the system 100.

A number of users 18 of recommendation system 100 may use interfaces of user devices 16 to exchange data and commands with servers 12 in manners described in further detail below. While three users 18 are shown in FIG. 1, recommendation system 100 is adaptable to be used by any suitable number of users 18, and even a single user 18. Furthermore, while recommendation system 100 shows two servers 10 and two databases 12, recommendation system 100 extends to any suitable number of servers 10 and databases 12 (such as a single server communicatively coupled to a single database).

In some embodiments, the function of databases 12 may be incorporated with that of servers 10 with non-transitory storage devices or memory. In other words, servers 10 may store the user data located on databases 12 within internal memory and may additionally perform any of the processing of data described herein. However, in the embodiment of FIG. 1, servers 10 are configured to remotely access the contents of databases 12 when required.

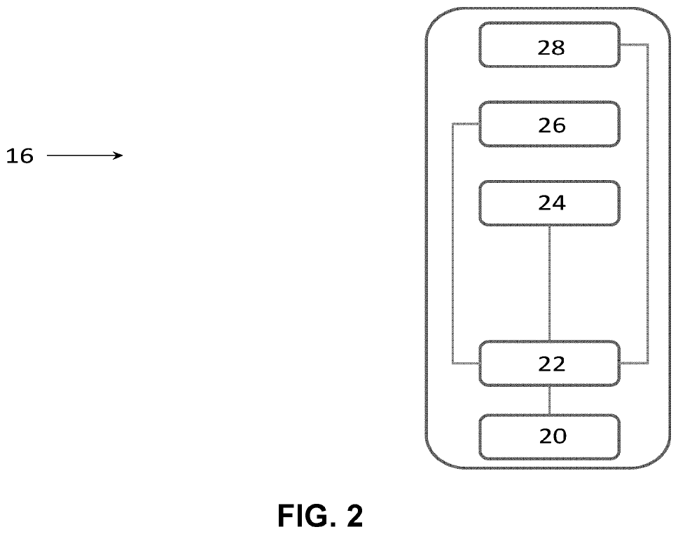

Turning to FIG. 2, there is shown an embodiment of a user device 16 in more detail. User device 16 includes a number of sensors, a hardware processor 22, and computer-readable medium 20 such as suitable computer memory storing computer program code. The sensors include a user interface 24, a camera 26, and a microphone 28, although the disclosure extends to other suitable sensors, such as biometric sensors (heart monitor, blood pressure monitor, skin wetness monitor etc.), any location/position sensors, motion detection or motion capturing sensors, and so on. The camera 26 can capture video and image data, for example. Processor 22 is communicative with each of sensors 24, 26, 28 and is configured to control the operation of sensors 24, 26, 28 in response to instructions read by processor 22 from non-transitory memory 20 and receive data from sensors 24, 26, 28. According to some embodiments, user device 16 is a mobile device such a smartphone, although in other embodiments user device 16 may be any other suitable device that may be operated and interfaced with by a user 18. For example, user device 16 may comprise a laptop, a personal computer, a tablet device, a smart mirror, a smart display, a smart screen, a smart wearable, or an exercise device.

Sensors 24, 26, 28 of user device 16 are configured to obtain user data relating to user 18. For example, microphone 28 may detect speech from user 18 whereupon processor 22 may convert the detected speech into voice data. User 18 may input text or other data into user device 16 via user interface 24, whereupon processor 22 may convert the user input into text data. Furthermore, camera 26 may capture images of user 18, for example when user 18 is interfacing with user device 16. Camera 26 may convert the images into image data relating to user 18. The user interface 24 can send collected data from the different components of the user device 16 for transmission to the server 10 and storage in the database 12 as part of data records that are stored with an identifier for the user device 16 and/or user 18.

The system 100 monitors one or more users over a user session using one or more sensors 24, 26, 28. In some embodiments, the system 100 provides an interface with activity recommendations for the user session, which can be user interface 24 of user device 16 in some embodiments, or an interface of a separate hardware device in some embodiments. The system 100 has non-transitory memory storing activity recommendation records, emotional signature records, and user records storing user data received from a plurality of channels, at servers 10 and databases 12, for example.

The user data can involve a range of data captured during a time period of the user session (which can be combined with data from different user sessions and with data for different users). The user data can be image data relating to the user, text input relating to the user, data defining physical or behavioural characteristics of the user, and audio data relating to the user.

The system 100 has a hardware processor (which can be at user device 16) programmed with executable instructions for an interface (which can be user interface 24 for this example) for obtaining user data for a user session over a time period. The processor transmits a recommendation request for the user session to the server 10, and updates its interface for providing activity recommendations for the user session received in response to the recommendation request.

The system 100 has a hardware server 10 coupled to the non-transitory memory (or database 12) to access the activity recommendation records, the emotional signature records, and the user records. The hardware server 10 is programmed with executable instructions to transmit the activity recommendations to the interface 24 over a network 14 in response to receiving the recommendation request from the interface. The hardware server 10 is programmed with executable instructions to compute the activity recommendations by: computing activity metrics, cognitive-affective competency metrics, and social metrics using the user data for the user session and the user records. The hardware server 10 can extract metrics from the user data to represent physical metrics of the user and cognitive metrics of the user. The hardware server 10 can use both physical metrics of the user and cognitive metrics of the user to determine the emotional signature for the user during the time period of the user session. The hardware server 10 can compute multiple emotional signatures for the user at time intervals during the time period of the user session. The hardware server 10 compute multiple emotional signatures which can trigger computation of updated activity recommendations and updates to the interface. The emotional signature uses both physical metrics of the user and cognitive metrics of the user during the time period of the user session.

The hardware server 10 can use user data captured during the user session and can also use user data captured during previous user sessions or user data for different users. The hardware server 10 can aggregated data from multiple channels to compute the activity recommendations to trigger updates to the interface 24 on the user device 16, or an interface on a separate hardware device in some examples.

The hardware server 10 can process different types of data by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis.

The hardware server 10 can compute one or more states of one or more cognitive-affective competencies of the user based on the cognitive-affective competency metrics and the social metrics. The hardware server 10 can compute an emotional signature of the user based on the one or more states of the one or more cognitive-affective competencies of the user and using the emotional signature records. The hardware server 10 can compute the activity recommendations based on the emotional signature of the user, the activity metrics, the activity recommendation records, and the user records. The system has a user device comprising one or more sensors for capturing user data during the time period, and a transmitter for transmitting the captured user data to the interface or the hardware server over the network to compute the activity recommendations.

In some embodiments, the system 100 has one or more modulators in communication with one or more ambient fixtures to change external sensory environment based on the activity recommendations, the one or more modulators being in communication with the hardware server 10 to automatically modulate the external sensory environment of the user during the user session. In some embodiments, the one or more ambient fixtures comprise at least one of a lightening fixture, an audio system, an aroma diffuser, a temperature regulating system.

The system 100 has multiple user devices 16 and each can have different types of sensors for capturing different types of user data during the user session. Each of the user devices 16 can be for transmitting the captured different types of user data to the hardware server 10 over the network 14 to compute the activity recommendations.

In some embodiments, the system 100 has multiple user devices 16 for a group of users. Each of the multiple user devices 16 has an interface for obtaining user data for a corresponding user of the group of users for the user session over the time period. The server 10 can provide activity recommendations for the user session received in response to recommendation requests from multiple user devices 16. The hardware server 10 transmits the activity recommendations to the corresponding interfaces 24 of the user devices 16 in response to receiving the recommendation request from the corresponding interfaces. The server 10 can compute activity recommendations for the group of users, can suggest activity recommendations based on user date for the group of users. The same activity recommendations can be suggested for all the users of the group, or a set of users of the group with similar emotional signatures as determined by the system 100 using similarity measurements.

In some embodiments, the hardware server 10 is configured to determine an emotional signature of one or more additional users and determine users with similar emotional signatures. The server 10 can predict connectedness between users with similar emotional signatures and generate the activity recommendations for the users with similar emotional signatures.

In some embodiments, the interface 24 of the user device 16 can receive feedback on the activity recommendations for the user session, transmit the feedback to the hardware server 10. The feedback can be positive indicating approval of the activity recommendations. The feedback can be negative indicating disapproval of the activity recommendations. The server 10 can use the feedback for subsequent computations of activity recommendations. The server 10 can store the feedback in the records of the database 12.

In some embodiments, the interface 24 can transmit another recommendation request for the user session, the server 10 can provide additional activity recommendations for the user session in response to the other recommendation request. The server 10 can transmit the additional activity recommendations for the user session to update the interface 24.

In some embodiments, the interface 24 obtains additional user data after providing the activity recommendations for the user session, the additional user data captured during performance of the activity recommendations by the user. The server 10 can use the additional user data captured after providing the activity recommendations for the user session to re-compute the emotional signature of the user during the performance of the activity recommendations.

In some embodiments, the interface 24 transmits another recommendation request for another user session, and provides updated activity recommendations for the other user session received from the server 10 in response to the other recommendation request. The updated activity recommendations can be different that the activity recommendations.

In some embodiments, the interface 24 is a coaching application and the one or more recommended activities are part of a virtual coaching program for the user.

In some embodiments, the activity recommendations are pre-determined classes selected from a set of classes stored in the activity recommendation records. In some embodiments, the activity recommendations are a program with variety of content for the interface to guide user's interactions or experience for a prolong time. In some embodiments, the one or more activity recommendations are a program with variety of content to guide user's interactions or experience for a prolong time. In some embodiments, the program comprises two or more phases, each phase having a different content, intensity or duration.

In some embodiments, the server 10 can receive user data relating to one or more additional users from user devices 16 and determine, based on the processed user data, one or more states of one or more cognitive-affective competencies of the one or more additional users. The server 10 can determine an emotional signature of each of the one or more additional users and determine users with similar emotional signatures. The server 10 can predict connectedness between users with similar emotional signatures using similar models or measures stored in non-transitory memory. The server can generate one or more activity recommendations for transmission to interfaces of users with similar emotional signatures.

In some embodiments, the server 10 can determine, based on the processed user data, a personality type of the user, and determine the emotional signature of the user based on the personality type of the user. In some embodiments, the processed user data comprises personality type data, and the server 10 can determine the personality type of the user by comparing the personality type data to stored personality type data indicative of correlations between personality types and personality type data.

In some embodiments, the processed user data comprises cognitive-affective competency data, and the server 10 can determine the one or more states of the one or more cognitive-affective competencies of the user comprises: comparing the cognitive-affective competency data to stored cognitive-affective competency data indicative of correlations between states of cognitive-affective competencies and cognitive-affective competency data.

In some embodiments, the server 10 can determine at least one of: one or more mood states of the user, one or more attentional states of the user, one or more prosociality states of the user, one or more motivational states of the user, one or more reappraisal states of the user, and one or more insight states of the user. The server 10 can determine the one or more states of the one or more cognitive-affective competencies of the user based on the at least one of: the one or more mood states of the user, the one or more attentional states of the user, the one or more prosociality states of the user, the one or more motivational states of the user, the one or more reappraisal states of the user, and the one or more insight states of the user.

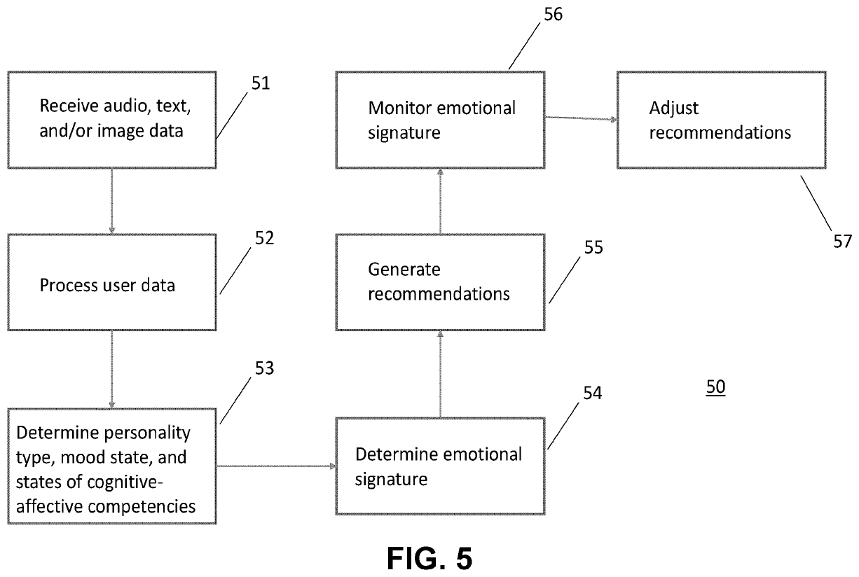

There will now be described a method 50 of generating recommendations for a user based on their emotional signature, and providing the recommendations via an interface. The method is shown generally in FIG. 5 which shows a flow diagram of the steps that may be taken to generate recommendations for a user, based on their emotional signature. As the skilled person would recognize, the steps shown in FIG. 5 are exemplary in nature, and the order of the steps may be changed, and steps may be omitted and/or added without departing from the scope of the disclosure.

The process begins, for example, by a user providing credentials to the user device 16 at user interface 24 to trigger activity recommendations and real-time data capture to improve their general emotional wellbeing at a current time period based on the real-time user data. The user activates on user device 16 an emotional wellbeing application (not shown) stored on memory 20 to trigger the user interface 24. The emotional wellbeing application invites the user to input user data to user device 16. At block 51, user device 16 receives the user data relating to the user from the user interface 24, which can be collected from different sensors 24, 26, 28 in real-time to provide input data for generating activity recommendations based on (near) real-time computation of the emotional wellbeing metrics based on the real-time data. For example, in response to activating emotional wellbeing application, the user may be prompted to complete a series of exercises and/or questionnaires, and the user interface 24 collects real-time user data throughout the series of exercises or other prompts. For example, a questionnaire may be presented to the user on user interface 24 and may require the user to answer one or more questions comprised in the questionnaire. Alternatively, the user may be prompted to speak out loud to discuss emotionally difficult events or how they feel about others in their life. The user interface 24 can collect the captured audio data for provision to the server 12. In other examples, with consent data obtained from the user interface 24, various forms of biometric data may be passively recorded throughout the user's day-to-day life as captured from different sensors 24, 26, 28 in real-time. Additionally, non-biometric data may also be recorded at user device 16, such as location data relating to the user. Such data may be processed to detect and quantify changes in levels of cognitive-affective competencies, and any other information used to measure the user's emotional signature, as described in further detail below.

The user may provide the answers, for example, via text input to user interface 24, or alternatively may speak the answers. Spoken answers may be detected by microphone 28 and utterances can be converted into audio data by processor 22. Prior to, during, or after the completion of the questionnaire, the emotional wellbeing application may send control commands to cause camera 26 to record images and/or video of the user. The images may comprise at least a portion of the user's body, at least a portion of the user's face, or a combination of at least a portion of the user's body and at least a portion of the user's face. The captured images are then converted into image data (which may comprise video data), which forms part of the overall user data that is received at user device 16.

The combination of audio data, text data, and image data, and any other data input to user device 16 and that relates to the user, may be referred to hereinafter as user data. Other suitable forms of data may be comprised in the user data. For example, the user data may comprise other observable data collected through one or more Internet of Things devices, social network data obtained through social network analysis, GPS or other location data, activity data (such as steps), heart rate data, heart rate variability data, data indicative of a duration of time spent using the user device or one or more specific applications on the user device, data indicative of a reaction time to notifications appearing on the user device, social graph data, phone log data, and call recipient data.

The server 10 can store the user data in records indexed by an identifier for the user, for example. The user device 16 can transmit captured user data to the server 10 for storage in database. In some embodiments, the user device 16 can pre-process the user data using the emotional wellbeing application before transmission to server 16. The pre-processing by the emotional wellbeing application can involve feature extraction from raw data, for example. The user device 16 can transmit the extracted features to server 10, instead of or in addition to the raw data, for example. The extracted features may be facilitate efficient transmission and reduce the amount of data transmitted between the user device 16 and server 10, for example.

According to some embodiments, in addition to user data being captured via the sensors 24, 26, 28 of user device 16, wearable sensors (e.g. a heart rate monitor, a blood pressure sensor) positioned on the user may provide additional data (such as the user's physical activity levels) and may be inputted to user device 16 and may form part of the user data received at user device 16.

At block 52, user device 16 transmits, over network 14, the user data to servers 10. Servers 10 then process the user data using different processing techniques. For example, servers 10 may process the image data using any of various facial and/or body analysis or modelling techniques known in the art or yet to be discovered. In addition, servers 10 may process voice data using any of voice analysis techniques (including tone analysis techniques) known in the art or yet to be discovered. In addition, servers 10 may process user input data (which may include audio data or text data) using different voice, text, social network, or behavioural analysis techniques (including tone analysis techniques and semantic analysis techniques) to extract features or metrics that can be used to compute a real-time emotional signature for the user. The real-time emotional signature can map to one or more activities that can be provided as recommendations to the user via user interface 24 and user device 16.

By processing the user data in this fashion, at block 53 servers 10 are able to identify one or more mood levels or states of the user. In addition to mood sensing (e.g. determining the user's current mood state), servers 10 are able to perform operations to compute different metrics corresponding to attention sensing (e.g. determining the user's external attentional deployment, internal attentional deployment, etc.), prosocial sensing (e.g. determining the user's emotional expression and behaviour with others, etc.) motivational state sensing, reappraisal state sensing, and insight state sensing. Such sensing techniques are examples of state detection sensing techniques that may be used to quantify an individual user's levels of cognitive-affective competencies, as well as determine the individual's personality type based on collected data.

For example, metrics or data corresponding to attention sensing may be determined by processing eye tracking data and through 3D modelling of the user's face and/or body, as well as the context or environment in which the user is in, and in addition to the object of attention or lack of such an object. Prosociality sensing relates to the detection of a user's positively/negatively valenced actions towards another person or towards themselves (e.g. giving a compliment, transmitting a positive/negative emotion such as smiling, mentioning a positive/negative action another person has undertaken, etc.).

Motivational sensing relates to computation of metrics based on the detection and distinction of the two subsystems of motivation known as the approach and avoid systems, which guide user behaviour based usually on a reward or a punishment (e.g. identifying a user's motivation through the way they describe their reason for completing a task, specific emotions displayed during a goal-oriented behaviour, etc.). Such motivation may be determined by processing the user's data input and activity data.

Reappraisal state sensing relates to computation of metrics based on detection of the user's recollection of an event and its affective associations, such associations being simultaneously weakened through active or passive means (e.g. having a user recall a difficult event over time and monitoring changes in emotional expression during the recollection). Extinction and reconsolidation can depend on numerous factors, such as level-of-processing, emotional salience, the amount of attention paid to a stimulus, the expectations at encoding regarding how memory will be assessed later, or the reconsolidation-mediated strengthening of memory trace. Extinction does not erase the original association, but is a process of novel learning that occurs when a memory (explicit or implicit) is retrieved and the constellation of conditioned stimuli that were previously conditioned to elicit a particular behavior or set of behavioral responses is temporarily labile and the associations with each other are weakened through active or passive means. Such recollection may be determined by processing the user's data input, biometric data, and historic emotional signatures and associated recommendations.

Insight sensing relates to the computation of metrics based on realization of “no-self” or non-attachment, which is a distinction between the phenomenological experience of oneself and one's thoughts, emotions, and feelings that appear “thing-like” and is described as a “release from mental fixations”. Insight sensing also relates to decentering which introduces a “space between one's perception and response” allowing the individual to disengage or “step outside” one's immediate experience in an observer perspective for insight and analysis of one's habitual patterns of emotion and behavior. Insight sensing may detect moments when an individual is relating to their thoughts, feelings, emotions, or bodily sensations as separate from who they are through how they are describing their experience and other non-verbal cues. Such sensing may be determined by processing the user's data input, biometric data, and historic emotional signatures and associated recommendations.

A personality type of the user may be generally estimated by metrics that correspond to values for one or more states or levels of any of various different models of personality types, such as the five-factor model: openness/intellect, conscientiousness, extraversion, agreeableness, and neuroticism/emotional stability. A mood state of the user may be determined by computed metrics that include one or more indications of: amusement, anger, awe, boredom, confusion, contempt, contentment, coyness, desire, disgust, embarrassment, fear, gratitude, happiness, interest, love, pain, pride, relief, sadness, shame, surprise, sympathy, and triumph. Cognitive-affective competencies of the user may include one or more of: intention and motivation, attention regulation, emotion regulation, memory extinction and reconsolidation, prosociality, and non-attachment and decentering. Cognitive-affective competencies are described more generally in “The Specific Affect Coding System (SPAFF)”, Coan, J. A., et al. (2001), Handbook of Emotion Elicitation and Assessment (pp. 106-123), New York, N.Y., the entirety of which is hereby incorporated by reference.

The automated detection/recognition of emotional characteristics in a person can be determined by processing the user data to extract and evaluate features relevant to emotional characteristics from the user data. The following examples are hereby incorporated by reference in their entirety:

“Detection and Analysis of Emotion from Speech Signals”, Davletcharova, A., et al., Procedia Computer Science Volume 58, 2015, Pages 91-96;

“Emotion Recognition from Facial Expressions using Images with Pose, Illumination and Age Variation for Human-Computer/Robot Interaction”, Palaniswamy, S., et al. (2018), Journal of ICT Research and Applications 12(1):14 Apr. 2018;

“Emotion recognition from skeletal movements”, Sapinsky, T., et al. (2019), Entropy, 21 (7), 646;

“Emotion Recognition through Gait on Mobile Devices”, Chiu, M., et al., EmotionAware'18—2nd International Workshop on emotion awareness for pervasive computing with mobile and wearable devices;

“The effect of emotion on movement smoothness during gait in healthy young adults”, Kang, G. E., et al., J Biomech. 2016 Dec. 8; 49(16):4022-4027;

“Automatic Emotion Perception Using Eye Movement Information for E-Healthcare Systems”, Wang, Y., et al., Sensors (Basel). 2018 September; 18(9): 2826;

“Identifying Emotional States using Keystroke Dynamics”, Epp, C., et al., CHI 2011 Session: Emotional States, May 7-12, 2011 Vancouver, BC, Canada;

“Towards affective touch interaction: predicting mobile user emotion from finger strokes”, Shah, S., et al., Journal of Interaction Science December 2015, 3:6; and

“Analysis of Facial EMG Signal for Emotion Recognition Using Wavelet Packet Transform and SVM”, Kehri, V., et al., Machine Intelligence and Signal Analysis pp 247-257.

At block 54, based on the determined personality type and states of cognitive-affective competencies of the user, an emotional signature of the user is determined by the server 10 using data received from the user device 16. According to some embodiments, the emotional signature is a combination of the data values corresponding to the determined personality type and states of cognitive-affective competencies of the user by the server 10 access the user data stored in databases 12 as captured by the user device 16 in (near) real-time. The emotional signature may act as a unique metric (or combinations of metrics) identifying the current overall emotional wellbeing of the user.

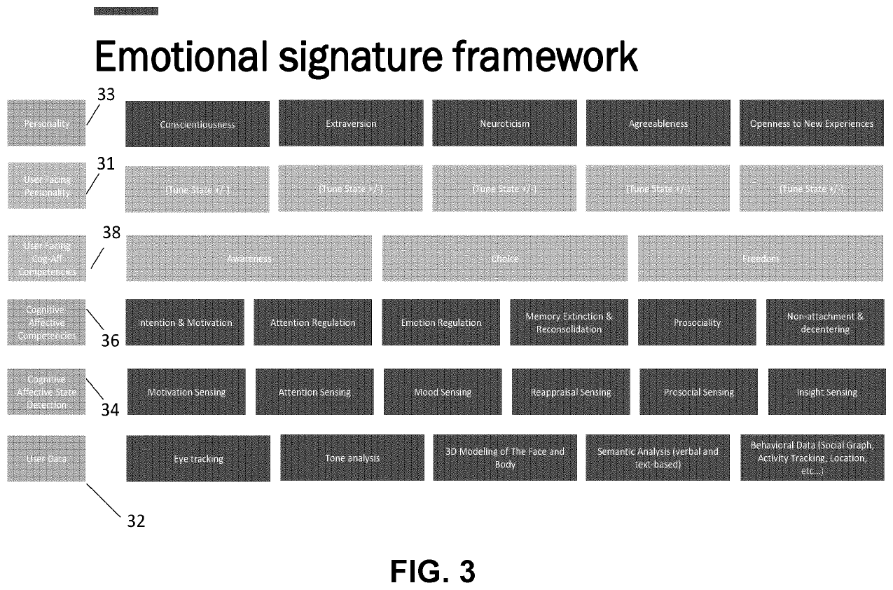

FIG. 3 shows an example relationship between different user data and how it relates to cognitive-affective competencies and personality types. The framework can be stored at server 10 (e.g. in database 12) as code instructions and data records that map parameters for cognitive-affective competencies and personality types to user data. In row 32, there are shown different forms of algorithms, techniques, and analysis processes for determining user data relating to the user. In row, 34, there are shown different types of cognitive-affective state detections methods, based on the type of user data that is captured. The server 10 can implement different types of cognitive-affective state detections methods, detects the type of user data that is captured, and selects the appropriate type of cognitive-affective state detections method for processing the user data. For example, eye tracking data may enable recommendation system 100 to sense a level of a user's attention, whereas 3D modelling and analysis of the user's face and body may enable recommendation system 100 to sense one or more moods of the user. In rows 36 and 38, there are shown different types of cognitive-affective competencies. In rows 31 and 33, there are shown different levels or states of different aspects of a user's personality type.

At block 55, servers 10 generate one or more recommendations in response to computing data for the emotional signature of the user. The servers 10 can generate activity recommendations. For example, a recommendation may comprise a recommendation to access or use particular content, coaches, events, groups, platonic/romantic matches, or other social or emotional learning experiences, for improving the user's emotional signature. For example, an emotional signature may indicate that the user is having difficulty in disrupting negative mental rumination as a result of low levels of decentering and non-attachment. In response, the recommendations may include consuming content (e.g. video, audio, one-on-one therapy) aimed at teaching a particular meditation which focuses on decentering.

The recommendations generated by recommendation system 100 and outputted to the user on their user device 16 may take the form of a training program to be executed by the user. For example, the training program may comprise one or more microcycle phases (daily-weekly programming), one or more mesocycle phases (2-6 week programming), and one or more macrocycle phases (annual programming). The intensity and volume of the training sessions may be varied linearly or non-linearly. While the levels of cognitive-affective competencies may vary over time, they are generally trainable. Thus, through repeated interventions (e.g. meditation), the propensity for a person to process, for example, emotional stimuli in a negative or positive way may change based on training duration and consistency.

The recommendation system 100 can store data for activity recommendations in database 12 and server 10, and generate the recommendations by identifying one or more activity recommendations from the stored data. For example, the recommendations may be generated based on known recommendations stored in association with known personality types and states of cognitive-affective competencies. Such associations between known recommendations and known personality types and states of cognitive-affective competencies may be stored, for example, in databases 12, and may be accessed by servers 10.

Over time, through repeated interaction of the user with the emotional wellbeing application on their user device 16, the emotional signature of the user may be tracked or monitored (block 56 of method 50). The recommendation system 100 will continue to receive user data in real-time from the user device 16 to re-compute the emotional signature of the user based on the updated user data. The recommendation system 100 can continuously collect user data and re-compute the emotional signature. For example, after the user has carried out the recommendations generated at block 55, the user may repeatedly or regularly interface with emotional wellbeing application to obtain or capture additional user data that is used to compute an updated emotional signature. The updated emotional signature may be compared by the server 10 to the last known or computed emotional signature of the user. If the updated emotional signature shows improvement, then the particular recommendations that the user performed may be understood as being beneficial for any other users having similar emotional signatures.

The server 10 may determine that an emotional signature shows improvement if, for example, the levels of cognitive-affective competencies comprised in the emotional signature of the user have beneficially increased, for instance if the mood state of the user is repeatedly assessed to be positive. On the other hand, an emotional signature may show deterioration if, for example, the levels of cognitive-affective competencies comprised in the emotional signature of the user have negatively decreased, for instance if the mood state of the user is repeatedly assessed to be negative. A deterioration in the emotional signature of a user may be indicative that the recommendations carried out by the user are not effectively improving the user's overall emotional wellbeing, and that alternative recommendations may be required. In such cases, recommendation system may (at block 57) adjust the recommendations that are generated in response to determining the updated emotional signature of the user and determining that the updated emotional signature has deteriorated relative to the last known emotional signature of the user.

Particular emotional signatures may therefore be associated with particular recommendations that have been shown to improve those emotional signatures over time. Such associations, or data indicative of such associations, may be stored for example in databases 12 for future use, and may be accessed by servers 10 when determining the recommendations to generate for a user. Accordingly, when a new emotional signature for the user session or a new user session is established for a user of recommendation system 100, servers 10 may access databases 12 to identify a recommendation or recommendations that have been shown to result in an improvement to similar emotional signatures for other users of recommendation system 100.

As an example, a user Jonathan decides to use recommendation system 100 to determine his emotional signature by providing user data to the server 10 via the user device 16. Based on the information provided by Jonathan to his user device 16, and based on an analysis of the user data, including user data representing Jonathan's facial expressions, body language, tone of voice, measured biometrics, and behavioural patterns (based on text input provided by Jonathan in response to questions posed by the emotional wellbeing application), recommendation system 100 determines that Jonathan's emotional signature is similar to the emotional signature of Alice (another user). Recommendation system 100 recently (e.g. in a previous user session or as part of the same user session) recommended to Alice that she spend more time in the outdoors (e.g. recommended activity involved nature), as Alice's emotional signature indicated a positive correlation between her mood and how much of her time was spent in nature outside. Over time, by repeatedly interfacing with the emotional wellbeing application to provide updated user data, Alice's emotional signature as computed by recommendation system 100 showed improvement as a result of spending more time in the outdoors. Recommendation system 100 therefore makes the same recommendation to Jonathan, given his similar emotional signature to the emotional signature of Alice.

By generating and monitoring an emotional signature for each user, recommendation system 100 is able to build a dataset of emotional signatures (stored as emotional signature records) and corresponding recommendations that are likely to improve individual emotional signatures.

Additionally, recommendation system 100 may enable individual users with similar emotional signatures to be put in contact with one another, for example by providing access to relevant contact information. According to some embodiments, recommendation system 100 may be used by team leaders, for example managers, in forming suitable teams. For instance, recommendation system 100 may be used to identify individuals that have similar emotional signatures and that may therefore work more efficiently or collaborate better when placed in the same team. The system 100 can establish a communication session between multiple user devices 16, for example.

According to some embodiments, recommendation system 100 may be configured to match people based on their emotional signature, such that the matching persons can develop deep and meaningful romantic or friendship relationship, or the recommendation system 100 may be used to match a person with a coach or to a matching content. The recommendation system 100 may be used to identify individuals that have similar emotional signatures and therefore may connect on a deep and meaningful way. For example, based on users' input data (facial analysis, voice analysis, body analysis, textual input, activity input, biometrics input, etc.), as well as user's levels of cognitive-affective competencies and the individual's personality type, the recommendation system 100 can identify connections that may turn into a multi-year relationship or recommend activities that involve a compatible community or coaches that have high probability of long lasting connections between users and improved wellbeing.

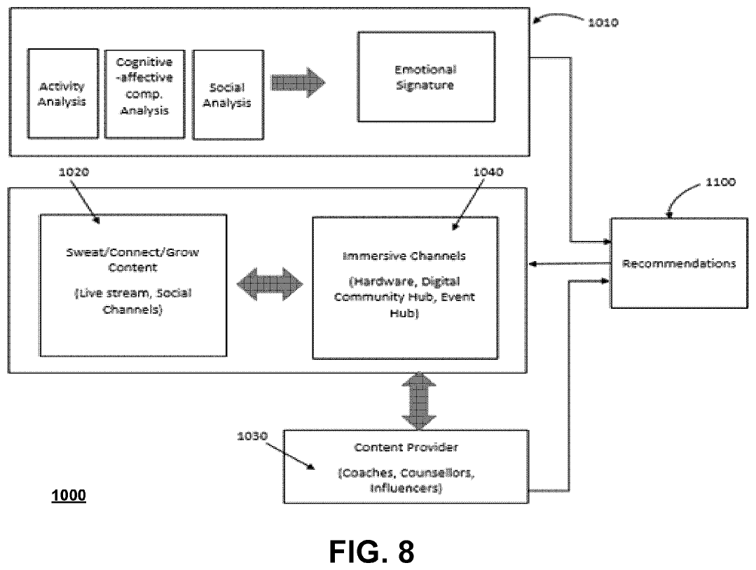

FIG. 8 illustrates an example of a wellbeing system 1000 that uses recommendation system 1100 to match users to a certain (recommended) activities content to improve user's wellbeing. The wellbeing system 1000 aggregates and processes user data across multiple channels to extract metrics for determining an emotional signature to provide improved activity recommendations and trigger effects for a user's environment by actuating sensory actuators to impact the sensory environment for the user. The wellbeing system 1000 has a wellbeing application 1010 with a hardware processor having an interface to display recommendations derived based on user data, activity metrics, and an emotional signature of a user computed by the hardware processor accessing memory storing the user data and extracted metrics. The wellbeing application 1010 receives user data from multiple channels, such as different hardware devices, digital communities, events, live streams, and so on. The wellbeing application 1010 has hardware processors that can implement different data processing operations to extract activity metrics, cognitive-affective competency metrics, and social metrics by processing the user data from different channels.

The wellbeing application 1010 stored on the non-transitory memory 20 of the user device 16 along with the sensors 24-28 receives user input and, according to the method described herein before, processes the user input to determine the emotional signature of such user. The wellbeing application 1010 can also connect to a separate hardware server (e.g. 1100) to exchange data and receive output data used to generate the recommendations or determine the emotional signature.

The wellbeing system 1000 can receive input data from different data sources or channels, such as different content providers 1030 (i.e., coaches, counsellors, influencers). The wellbeing system 1000 can aggregate and store content into a content center 1020. As new input data is collected over an updated time period, the wellbeing system 1000 can recompute updated emotional signatures. Based on user's emotional signature, the recommendation system 1100 may suggest, for example, content activity provided by a matching coach, to help to improve user's wellbeing and/or achieve his/her goals. During performance of the activities, the wellbeing system 1000 can receive data indicating user's performance from a data stream from an immersive hardware device (channels 1040), such as for example, a smart watch, a smart phone, a smart mirror, or any other smart exercise machine (e.g., connected stationary bike) as well as any other sensors, such as sensors 24-26. Based on the collected data and user's emotional signature, the recommendation system 1100 can dynamically adapt the user's activities and/or goals. In one implementation, the recommendations generated by recommendation system 1100 may take the form of a program to guide or shape matching pair/community interactions or experience. For example, the program may comprise one or more phases (daily, weekly, monthly, yearly programming). A program can be a series of activities that can map to time segments or intervals during the time period of the user session. Different activities and sessions may be recommended based on the phase. The system 1000 can map activity data to phases. The intensity and volume of the sessions and activities recommended may be varied linearly or non-linearly. Over time, through repeated interaction of the users with the emotional wellbeing application on their user device 16, updated user data is captured by the recommendation system 1100 and the emotional signature of each user may be tracked or monitored based on the updated user data collected over time. The recommendation system 1100 may change the recommendations in the program based on the current emotional signatures of the matching persons to maintain deep meaningful connections between the matched users. The system 1100 can compute updated emotional signatures at different intervals over the time period of a user session.

The emotional signature can be a data structure of values (stored as records in non-transitory memory accessible by a hardware processor) that the system 1000 can compare to other data structures of values representing other emotional signatures using different similarity measures or functions, for example. Different similarity measures can be used to identify similar emotional signatures.

In some implementations, the methods and systems described herein can use user's emotional signatures to make activity class recommendations. Group exercises improve individual well being and increase social bonding through shared emotions and movement. Therefore, the recommendation system 1100 may be used to identify individuals that have similar emotional signatures and to connect them by recommending a class content or event and matching class/event peers. The recommendation system 1100 can also generate social metrics for the user to make recommendations.

In some implementations, the wellbeing system 1000 may manipulate external sensory environment (such as sound, lighting, smell, temperature, air flow in a room) to alter an individual's (or group of individuals) interoceptive ability to deliver greater physiological and psychological benefits during the class/experience. The system 1000 can manipulate the external sensory environment based on the activity inputs (e.g., type of activity, content, class intensity, class durations) received at user device 16, biometric inputs of users measured in real time during the class using the user device 16, as well as users' individual emotional signatures calculated by the system 1000 during previous sessions. For example, based on the emotional signature of the user or group of users, the recommendation system 1100 may recommend a class or activity to such user or group of users and then the sound tempo or volume can be altered to match the recommended class/activity, such as sequence of movements as well as user biometric input obtain during class/activity, such as for example, cadence of the user or group of users. Depending of recommended activity and the emotional signature of the user or group of users, the wellbeing system 1000 can dynamically change the external sensory environment during the duration of the activity or experience to match the sequence/intensity of the activity/experience as well as users biometrics, or visual or audio cues/inputs.

The wellbeing application 1010 can use different data processing techniques to generate the emotional signature. For example, the wellbeing application 1010 can receive data sets (e.g. that can be extracted from aggregated data sources), extract metrics from the aggregated data sources, and generate the emotional signature for improved wellbeing using the extracted insights. The wellbeing application 1010 can transmit the emotional signature to the recommendation system 1100. An interface can connect to the recommendation system 1100 to display visual effects based on the emotional signature. An interface can connect to the recommendation system 1100 to display the generated recommendation, or trigger updates to the interface based on the recommendation (e.g. change an activity provided by the interface).

The system 1000 monitors one or more users over a user session using one or more sensors. In some embodiments, the wellbeing application 1010 has an interface providing activity recommendations for the user session. The system 1000 has non-transitory memory storing activity recommendation records, emotional signature records, and user records storing user data received from a plurality of channels 1040, for example.

The user data can involve a range of data captured during a time period of the user session (which can be combined with data from different user sessions and with data for different users). The user data can be image data relating to the user, text input relating to the user, data defining physical or behavioural characteristics of the user, and audio data relating to the user.

The wellbeing application 1010 resides on a hardware processor (which can be at user device 16) programmed with executable instructions for an interface for obtaining user data for a user session over a time period. The wellbeing application 1010 transmits a recommendation request for the user session to recommendation system 1100, and updates its interface for providing activity recommendations for the user session received in response to the recommendation request.

The wellbeing application 1010 can be coupled to non-transitory memory to access the activity recommendation records, the emotional signature records, and the user records.

The recommendation system 1100 is programmed with executable instructions to transmit the activity recommendations to the wellbeing application 1010 over a network in response to receiving the recommendation request. The wellbeing application 1010 is programmed with executable instructions to compute the activity recommendations based on metrics received by wellbeing application 1010 in this example embodiment. The wellbeing application 1010 can compute activity metrics, cognitive-affective competency metrics, and social metrics using the user data for the user session and the user records. The wellbeing application 1010 can extract metrics from the user data to represent physical metrics of the user and cognitive metrics of the user. The wellbeing application 1010 can use both physical metrics of the user and cognitive metrics of the user to determine the emotional signature for the user during the time period of the user session. The wellbeing application 1010 can compute multiple emotional signatures for the user at time intervals during the time period of the user session. The wellbeing application 1010 compute multiple emotional signatures which can trigger computation of updated activity recommendations and updates to the interface. The emotional signature uses both physical metrics of the user and cognitive metrics of the user during the time period of the user session.

The wellbeing application 1010 can transmit the computed emotional signatures to the recommendation system 1100 to as part of the request, for example. The recommendation system 1100 can use user data captured during the user session and can also use user data captured during previous user sessions or user data for different users. The recommendation system 1100 can aggregated data from multiple channels to compute the activity recommendations to trigger updates to the wellbeing application 1010, or an interface on a separate hardware device in some examples.

The recommendation system 1100 can process different types of data by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis.

The wellbeing application 1010 can compute one or more states of one or more cognitive-affective competencies of the user based on the cognitive-affective competency metrics and the social metrics. The wellbeing application 1010 can compute an emotional signature of the user based on the one or more states of the one or more cognitive-affective competencies of the user and using the emotional signature records. The recommendation system 1100 can compute the activity recommendations based on the emotional signature of the user, the activity metrics, the activity recommendation records, and the user records.

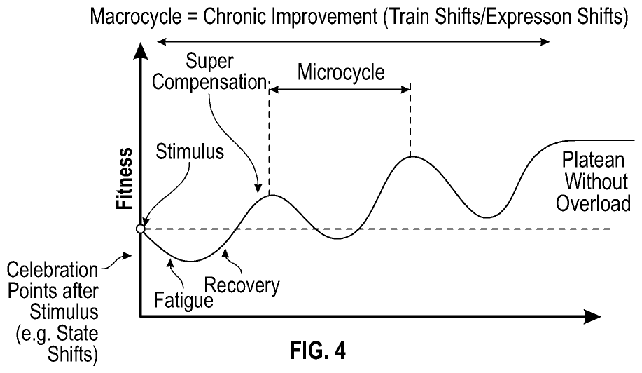

FIG. 4 shows an example improvement in a person's emotional fitness or wellbeing over a period of time, in response to the execution of one or more of various recommendations generated by recommendation system 100 as a result of identifying the user's particular emotional signature. This is so called “periodization” in physical fitness training and subsequent improvement in fitness, wherein the periodization being the process of systematic planning of training. So, in addition to being used to display improvement over time, it can also be a tool of coaching in terms of planning out when emotional fitness content, training or interventions should be delivered to the user, in order to make sure they improve over time (instead of getting worse). The coach would plan the cycles of training (meso and macrocycles) to ensure the user gets enough content to challenge and engage them, but not too much so that they feel overwhelmed.

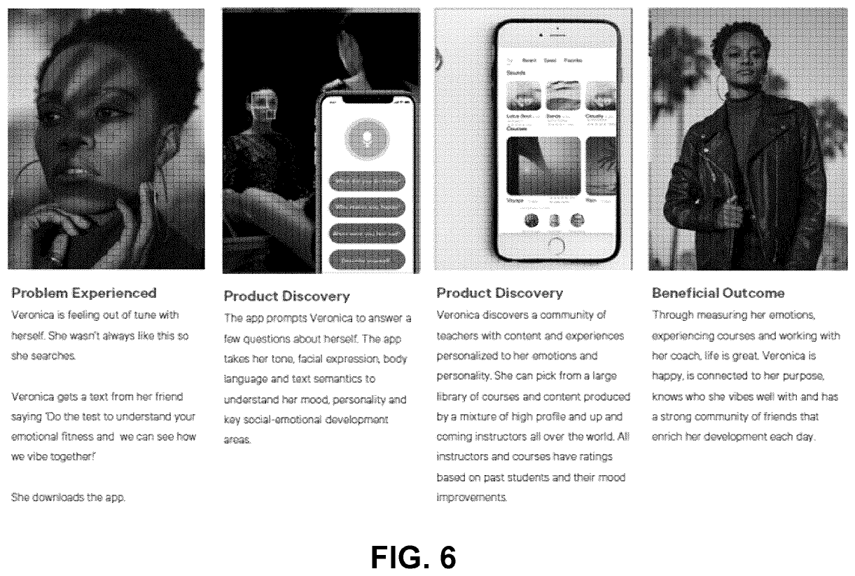

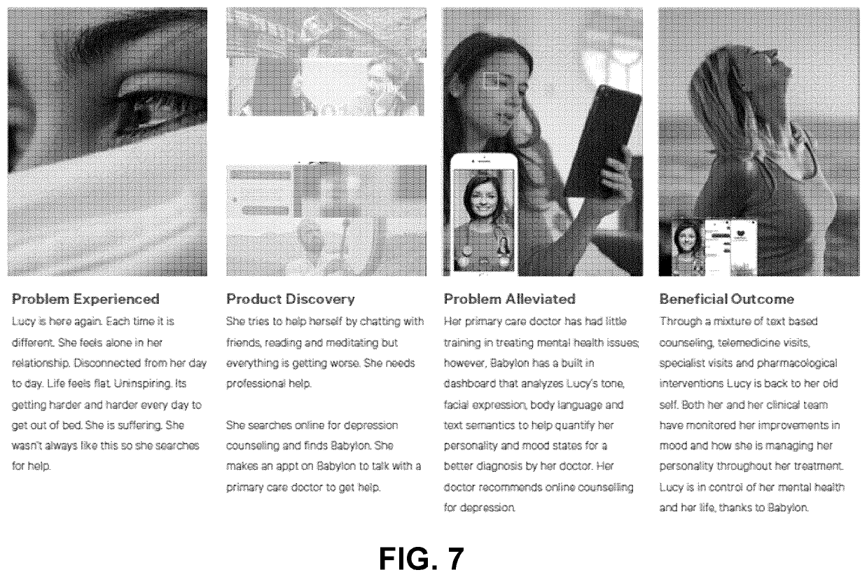

FIGS. 6 and 7 illustrate the improvement of the emotional signatures of different users, according to example embodiments of the disclosure.

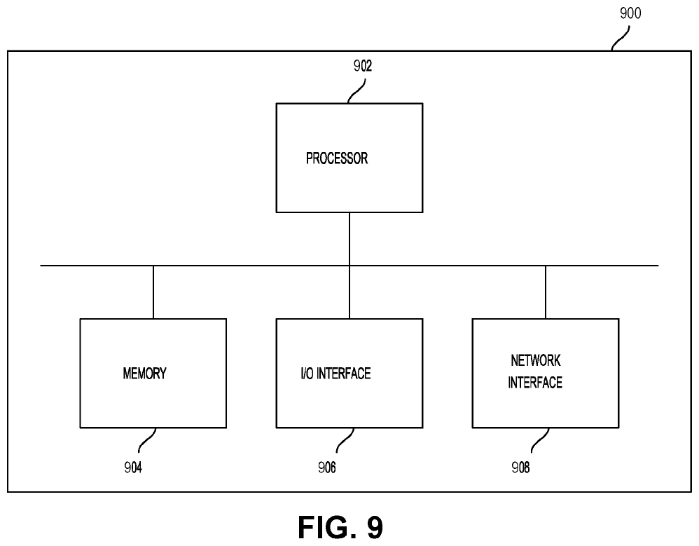

FIG. 9 shows an example schematic diagram of a computing device 900 that can implement aspects of embodiments, such as aspects or components of user device 16, servers 10, databases 12, system 1100, or application 1010. As depicted, the device 900 includes at least one hardware processor 902, non-transitory memory 904, and at least one I/O interface 906, and at least one network interface 908 for exchanging data. The /0 interface 906, and at least one network interface 908 may include transmitters, receivers, and other hardware for data communication. The I/O interface 906 can capture user data for transmission to another device via network interface 908, for example.

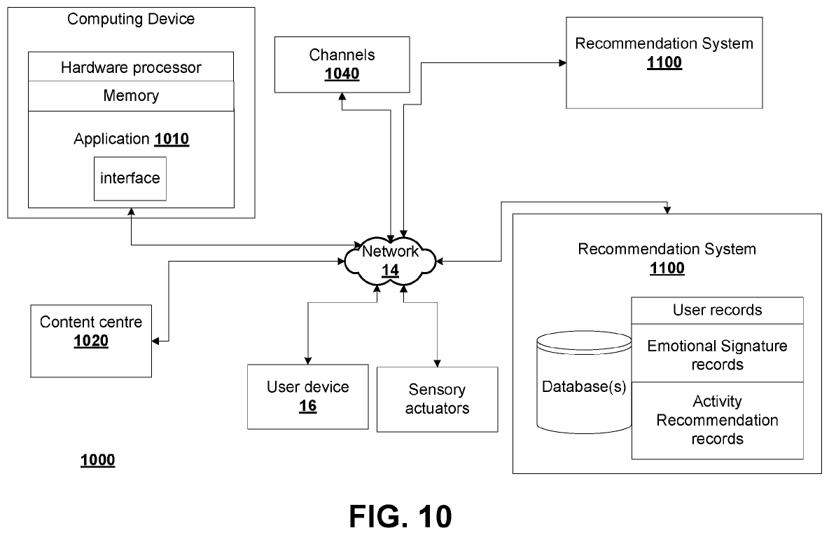

FIG. 10 illustrates another example of a wellbeing system 1000 with a wellbeing application 1010 that uses recommendation system 1100 to provide activity recommendations based on user data captured across the distributed system 1000. The recommendation system 1100 and/or wellbeing application 1010 can receive input data from different data sources, such as content center 1020, user devices 16, and channels 1040.

The system 1000 monitors one or more users over a user session using user device 16 with sensors. In some embodiments, the wellbeing application 1010 has an interface with activity recommendations for the user session. The recommendation system 1100 has non-transitory memory storing activity recommendation records, emotional signature records, and user records storing user data received from a plurality of channels 1040, for example.

The user data can involve a range of data captured during a time period of the user session (which can be combined with data from different user sessions and with data for different users). The user data can be image data relating to the user, text input relating to the user, data defining physical or behavioural characteristics of the user, and audio data relating to the user.

The wellbeing application 1010 resides on a hardware processor (which can be at user device 16 or a separate computing device) programmed with executable instructions for an interface for obtaining user data for a user session over a time period. The wellbeing application 1010 transmits a recommendation request for the user session to the recommendation system 1100, and updates its interface for providing activity recommendations for the user session received in response to the recommendation request.

The recommendation system 1100 is programmed with executable instructions to transmit the activity recommendations to the interface of wellbeing application 1010 over a network 14 in response to receiving the recommendation request from the interface. The recommendation system 1100 is a hardware server 10 programmed with executable instructions to compute the activity recommendations by: computing activity metrics, cognitive-affective competency metrics, and social metrics using the user data for the user session and the user records. The recommendation system 1100 can extract metrics from the user data to represent physical metrics of the user and cognitive metrics of the user. The recommendation system 1100 can use both physical metrics of the user and cognitive metrics of the user to determine the emotional signature for the user during the time period of the user session. The recommendation system 1100 can compute multiple emotional signatures for the user at time intervals during the time period of the user session. The recommendation system 1100 compute multiple emotional signatures which can trigger computation of updated activity recommendations and updates to the interface. The emotional signature uses both physical metrics of the user and cognitive metrics of the user during the time period of the user session.

The recommendation system 1100 can use user data captured during the user session and can also use user data captured during previous user sessions or user data for different users. The recommendation system 1100 can aggregated data from multiple channels to compute the activity recommendations to trigger updates to the interface.

The recommendation system 1100 can process different types of data by: for the image data and the data defining the physical or behavioural characteristics of the user, using at least one of: facial analysis; body analysis; eye tracking; behavioural analysis; social network or graph analysis; location analysis; user activity analysis; for the audio data, using voice analysis; and for the text input using text analysis.

The recommendation system 1100 can compute one or more states of one or more cognitive-affective competencies of the user based on the cognitive-affective competency metrics and the social metrics. The recommendation system 1100 can compute an emotional signature of the user based on the one or more states of the one or more cognitive-affective competencies of the user and using the emotional signature records. The recommendation system 1100 can compute the activity recommendations based on the emotional signature of the user, the activity metrics, the activity recommendation records, and the user records. The system has a user device comprising one or more sensors for capturing user data during the time period, and a transmitter for transmitting the captured user data to the interface or the hardware server over the network to compute the activity recommendations.

The wellbeing application 1010 has an interface that receives a recommendation request, transmits the request to the recommendation system 1100, and updates its interface to provide an activity recommendation in response to the request. The wellbeing application 1010 has the interface to provide the recommendations derived based on user data, activity metrics, and an emotional signature of a user.

The recommendation request can relate to a time period and the activity recommendation generated in response to the request can relate to the same time period. In some embodiments, the wellbeing application 1010 can determine the activity recommendation. The wellbeing application 1010 has an interface that can display the activity recommendation or otherwise provide the activity recommendation such as by audio or video data. The wellbeing application 1010 is shown on a computing device with a hardware processor in this example.

In some embodiments, the wellbeing application 1010 can transmit the recommendation request to the recommendation system 1100 to determine an activity recommendation. The wellbeing application 1010 can transmit additional data relating to the recommendation request such as a time period, user identifier, application identifier, or captured user data to the recommendation system 1100 to receive an activity recommendation in response to the request.

The wellbeing application 1010 can process the user data to determine the emotional signature of such user, or the wellbeing application 1010 can communicate with the recommendation system 1100 to compute the emotional signature. The recommendation system 1100 can use the emotional signature for the user for the time period to generate the activity recommendation for the wellbeing application 1010.

For example, in some embodiments, the wellbeing application 1010 can determine an emotional signature of the user for the time period, and send the emotional signature for the time period to the recommendation system 1100 along with the recommendation request. The wellbeing application 1010 can store instructions in memory to determine the emotional signature for a user for a time period. The wellbeing application 1010 is shown on a computing device with non-transitory memory and a hardware processor executing instructions for the interface to obtain user data and provide activity recommendations. For example, the wellbeing application 1010 can obtain user data by connecting to a user device 16 along with the sensors 24-28 collecting the user data for a time period. The wellbeing application 1010 can connect to the separate hardware server (e.g. recommendation system 1100) to exchange data and receive output data used to generate the recommendations or determine the emotional signature.

The wellbeing application 1010 can obtain user data from the multiple channels 1040, or collect user data from user device 16 (with sensors) for computing the emotional signature. In other embodiments, the recommendation system 1100 determines the emotional signature of the user for the time period in response to receiving the recommendation request from the wellbeing application 1010. Using the recommendation system 1100 to compute the emotional signature for the user for the time period can offload the computation of the emotional signature for the user for the time period (and required processing resources) to the recommendation system 1100 which might have greater processing resources than the wellbeing application 1010, for example. The recommendation system 1100 can have secure communication paths to different sources to aggregated captured user data from different sources, to offload data aggregation operations to the recommendation system 1100 which might have greater processing resources than the wellbeing application 1010, for example.

In some embodiments, the wellbeing application 1010 can capture user data (via I/O hardware or sensors of computing device) for use in determining the emotional signature of the user for the time period and the activity recommendation. In some embodiments, one or more user devices 16 capture user data for use in determining the activity recommendation. In some embodiments, the wellbeing application 1010 can reside on the user device 16, or the wellbeing application 1010 can reside on a separate computing device than the user device 16.

In some embodiments, the wellbeing application 1010 can transmit the captured user data to the recommendation system 1100 as part of the recommendation request, or in relation thereto. In some embodiments, the wellbeing application 1010 extracts activity metrics, cognitive-affective competency metrics, and social metrics by processing captured user data. The captured user data can be distributed across different devices and components of the system 1000. The wellbeing application 1010 can receive and aggregate captured user data from multiple sources, including channels 1040, content centre 1020, user device 16, and recommendation system 1100. In some embodiments, the wellbeing application 1010 can extract activity metrics, cognitive-affective competency metrics, and social metrics by processing user data from multiple sources, and provide the extracted metrics to the recommendation system 1100 to compute the emotional signature and activity recommendations.

In some embodiments, in response to receiving the request from the wellbeing application 1010, the recommendation system 1100 can extract activity metrics, cognitive-affective competency metrics, and social metrics by processing captured user data for the time period. The recommendation system 1100 can register different applications 1010 to link an application identifier to a user identifier. The recommendation system 1100 can extract an application identifier from the request in some embodiments, to locate a user identifier to retrieve relevant records.

The recommendation system 1100 can receive and aggregate captured user data from multiple sources, including channels 1040, content centre 1020, user device 16, and application 1010. In response to receiving the request from the wellbeing application 1010, the recommendation system 1100 can request additional captured user data relevant to the time period from different sources. The recommendation system 1100 can use the aggregated user data from the multiple sources to extract activity metrics, cognitive-affective competency metrics, and social metrics by processing the captured user data for the time period. The user data from the multiple sources can be indexed by an identifier (e.g. user identification) so that the recommendation system 1100 can identify user data relevant to a specific user across different data sets, for example. The recommendation system 1100 has hardware processors that can implement different data processing operations to extract activity metrics, cognitive-affective competency metrics, and social metrics by processing the user data from different channels 1040, content centre 1020, user device 16, wellbeing application 1010. The recommendation system 1100 has a database or user records, emotional signature records, an activity recommendation records. The user records can store extracted activity metrics, cognitive-affective competency metrics, and social metrics for a user across different time periods, for example. The user records can store activity recommendations for a user for different time periods based on the extracted activity metrics, cognitive-affective competency metrics, and social metrics for the different time periods, for example.

The recommendation system 1100 uses the extracted activity metrics, cognitive-affective competency metrics, and social metrics to determine the activity recommendation for the time period. The recommendation system 1100 can extract activity metrics, cognitive-affective competency metrics, and social metrics, or can receive extracted activity metrics, cognitive-affective competency metrics, and social metrics from the wellbeing application 1010 (or different channels 1040, content centre 1020, user device 16), for example, or a combination thereof. The recommendation system 1100 can aggregate extracted activity metrics, cognitive-affective competency metrics, and social metrics for the user for the time period to determine the emotional signature of the user and the activity recommendation.

In some embodiments, the recommendation system 1100 aggregates user data from multiple sources (channels 1040, user device 16, content centre 1020) to leverage distributed computing devices so that the wellbeing application 1010 does not have to collect all the user data from all the different sources. The channels 1040, user device 16, content centre 1020 can have different hardware components to enable collection of different types of data. In some embodiments, the wellbeing system 1000 distributes the collection of user data across these different sources to efficiently collect different types of data from different sources. The recommendation system 1100 can have secure communication paths to different sources to aggregated captured user data from different sources in a secure way at a central repository, for example. Captured user data from multiple sources may contain sensitive data and the recommendation system 1100 can provide secure data storage. This can alleviate the need for the captured user data from multiple sources (with sensitive data) to be stored locally on different devices, which might create security issues, for example. This can offload data aggregation operations to the recommendation system 1100 which might have greater processing resources than the wellbeing application 1010, for example.

In some embodiments, the recommendation system 1100 computes the emotional signature for the user for the time period. The wellbeing application 1010 exchanges data with the recommendation system 1100 for computing the emotional signature. As noted, the recommendation system 1100 can send requests for updated user data, receive updated user data in response from multiple channels 1040, and aggregate the user data from the multiple channels 1040, such as different hardware devices, digital communities, events, live streams, and so on, for computing the emotional signature. The recommendation system 1100 can store the aggregated user data in user records, for example.

As new input data is collected by the recommendation system 1100 (or wellbeing application 1010, channels 1040, user device 16, content centre 1020) over an updated time period, recommendation system 1100 can compute an emotional signature for the user for the updated time period. If a new recommendation request is received by the recommendation system 1100 for an updated time period, the recommendation system 1100 can compute an emotional signature for the user for the updated time period. The emotional signature for the initial time period can be different from the emotional signature for the updated time period. The emotional signature for the updated time period is used to determine the activity recommendations. Accordingly, an updated emotional signature for the updated time period can trigger different activity recommendations than the activity recommendations determined based on the emotional signature for the previous time period.

In some embodiments, wellbeing application 1010 sends a request to the recommendation system 1100 to compute the emotional signature for the updated time period. In response, the recommendation system 1100 can compute a new emotional signature for the updated time period and can also determine new activity recommendations based on the emotional signature for the updated time period. In response, the recommendation system 1100 can send data for the emotional signature for the updated time period to the wellbeing application 1010, and can also send the new activity recommendations based on the emotional signature for the updated time period. Using the recommendation system 1100 for computation can offload processing requirements from the application 1010 to separate hardware processors of the recommendation system 1100.

The recommendation system 1100 stores data for the emotional signatures in a database of emotional signature records. Each emotional signature record can be indexed by a user identifier, for example. Each emotional signature record can indicate the time period, a value corresponding to the computed emotional signature for the time period, and extracted metrics, for example. The emotional signature record can also store any activity recommendations for the time period. The emotional signature records can include historic data about previous emotional signature determinations for the user for different time periods. The emotional signature records can include historic data about previous emotional signature determinations for all users of the system. The historic data for emotional signature records can include time data corresponding to time periods of user data used to compute emotional signatures. Accordingly, recommendation system 1100 can compute an emotional signature for a user for a time period and store the computed emotional signature in the database of emotional signature records with a user identifier, values for the computed emotional signature, and the time period. The emotional signature can be a data structure of values that the recommendation system 1100. The recommendation system 1100 can define parameters for the data structure of values that can be used to compute values for an emotional signature based on the captured user data for the time period. The recommendation system 1100 can compare to other data structures of values representing other emotional signatures using different similarity measures, for example. Different similarity measures can be used to identify similar emotional signatures. The recommendation system 1100 map emotional signatures (data structure of values) to user records and activity records.

In some embodiments, the recommendation system 1100 has a database of user records with user identifiers and user data. Each user record can be indexed by a user identifier, for example. The recommendation system 1100 can identify a set of emotional signature records based on a user identifier, for example, to identify emotional signatures determined for a specific user or to compare emotional signatures for a specific user over different time periods.

The recommendation system 1100 stores data for the activity recommendations in a database of activity recommendation records. Each activity recommendation can be indexed by an activity identifier, for example. The activity recommendation records can define different activities, parameters for the activities, identifiers for the activities, and other data. The activity recommendation records can include historic data about previous activity recommendations for the user, and previous activity recommendations for all users of the system. The historic data for activity recommendation records can include time data that can map to time periods of emotional signatures. A user record and/or a emotional signature record can also indicate an activity identifier to connect the user record and/or the emotional signature to a specific activity record. For example, the recommendation system 1100 can compute an emotional signature for a user for a time period based on user data, and determine an activity recommendation for the user for the time period. The activity recommendation can correspond to an activity recommendation record indexed by an activity identifier. The user record can store the activity identifier and the time period to connect the user record to a specific activity record. The emotional signature record might also indicate the user identifier, or an emotional signature identifier. The user record can also indicate the emotional signature identifier to connect the user record, the specific activity record, and the emotional signature record. An emotional signature record might also indicate parameters for computing different types of emotional signatures using different types of data. The emotional signature record might also have a model for computing a emotional signature for a time period. The emotional signature record might also indicate different activity identifiers to connect an emotional signature to an activity recommendation record.

Based on user's emotional signature, the recommendation system 1100 may transmit data to the application 1010 to update the interface. The data can be instructions for displaying new content on the interface or for generating audio or video data at the interface, for example.

The recommendation system 1100 and the application 1010 can connect using an application programming interface (API) and exchange commands (including the recommendation request) and data using the API. The application 1010 can receive instructions from the recommendation system 1100 to provide activity recommendations at the interface. For example, the application 1010 can provide a virtual coach interface that provides activity recommendations over time periods to help improve the user's wellbeing and/or achieve his/her goals. The application 1010 can exchange commands and data with the recommendation system 1100 using the API to receive activity recommendations and automatically update the virtual coach interface to automatically provide the activity recommendations. The application 1010 can use the virtual coach interface to prompt for user data, and can transmit collected user data to the recommendation system 1100 using the API.

The application 1010 can automatically update the interface to provide an activity recommendation for the time period. The application 1010 can continue to monitor the user (via collection of user data) during performance of the activity to collect feedback data, which can be referred to as user data. The application 1010 can receive positive or negative feedback about the activity recommendation for the time period. For example, the application 1010 updates the interface to provide a first activity recommendation for the time period and receives negative feedback about the first activity recommendation for the time period. The application 1010 can exchange commands and data with the recommendation system 1100 using the API to receive a second activity recommendation for the time period and communicate the negative feedback. The recommendation system 1100 can store the negative feedback in a user record with an activity identifier for the first activity recommendation for the time period, for example, or otherwise store the negative feedback in association with the first activity recommendation.

During performance of the activities, the wellbeing system 1000 can receive data indicating user's performance from a data stream from a different channels 1040 such as immersive hardware devices (as an example user device 16), such as for example, a smart watch, a smart phone, a smart mirror, or any other smart exercise machine (e.g., connected stationary bike) as well as any other sensors, such as sensors 24-26. For example, the user device 16 can be a smart mirror with a camera and sensors to capture user data. The user device 16 that is a smart mirror can also have the application 1010 with the interface, for example, to provide activity recommendations to the user for the time period. The application 1010 can send the recommendation request along with the captured user data from the user device 16 (smart mirror) to the recommendation system 1100 using the API to receive an activity recommendation for the time period to update the interface. Accordingly, the user device 16 have the application 1010 with the interface to provide activity recommendations for different time periods and also has sensors to capture user data for the time periods.

Based on the collected data and user's emotional signature, the recommendation system 1100 can dynamically adapt by providing updated activity recommendations over different time periods, or updated activity recommendations for the same time period based on feedback from the interface for previous activity recommendations. In one implementation, the recommendations generated by recommendation system 1100 may take the form of a program of multiple activity recommendations for a time period (or time segments) to guide or shape matching pair/community interactions or experience. For example, the program may comprise one or more phases of activity recommendations for different time periods (daily, weekly, monthly, yearly programming). The recommendation system 1100 can compute different activity recommendations and sessions based on the phase and current time period. The intensity and volume of the sessions and activities recommended may be varied linearly or non-linearly. Over time, through repeated interaction of the users with the emotional wellbeing application 1010 on their user device 16, updated user data is captured by the wellbeing application 1010 and sent to the recommendation system 1100 for tracking and storage. Over time, the recommendation system 1100 can track and monitor the emotional signature of each user based on the updated user data collected over time. The recommendation system 1100 may define a program as a set of activity recommendations. The recommendation system 1100 may change the program to change the set of activity recommendations. The recommendation system 1100 may change the program based on the current emotional signatures of the matching persons to align the set of activity recommendations to help maintain deep meaningful connections between the matched users.

The recommendation system 1100 can use emotional signatures to make activity class recommendations for a group of users. The recommendation system 1100 can generate the same activity recommendation for each user of the group, for example, based on the emotional signatures computed for each user of the group. Group exercises improve individual well being and increase social bonding through shared emotions and movement. Therefore, the recommendation system 1100 may be used to identify individuals that have similar emotional signatures and connect them by generating the same activity recommendation for a set of identified users or peers. Each user in the group can be linked to a wellbeing application 1010 and the recommendation system 1100 can send the same activity recommendation to each of the wellbeing applications 1010 for the set of identified users and continue to monitor the for a set of identified users by capturing additional user data after providing the same activity recommendation. The recommendation system 1100 can also generate social metrics for the user to make recommendations for the set of identified users.

In some implementations, the recommendation system 1100 may manipulate external sensory environment by controlling connected sensory actuators (such as sound, lighting, smell, temperature, air flow in a room). The sensory actuators can be part of a building automation system, for example, to control components of the building system. The recommendation system 1100 can transmit control commands to sensory actuators as part of the process of generating activity recommendations, computing emotional signatures, or ongoing monitoring of users by capturing additional user data.

The recommendation system 1100 may control connected sensory actuators to alter a user's (or group of users) interoceptive ability to deliver greater physiological and psychological benefits during the class/experience. The recommendation system 1100 can manipulate the connected sensory actuators based on the activity recommendations (e.g., type of activity, content, class intensity, class durations), feedback received at user device 16 or interface of wellbeing application 1010, biometric inputs of users measured in real time during the class using the user device 16, as well as users' individual emotional signatures calculated by the system 1000 during previous sessions.

For example, based on the emotional signature of the user or group of users, the recommendation system 1100 may generate an activity recommendation for such user or group of users and then sound tempo or volume related to the activity recommendation can be altered to match the recommended class/activity by the recommendation system 1100 controlling sensory actuators. Depending of recommended activity and the emotional signature of the user or group of users, the recommendation system 1100 can dynamically change the external sensory environment during the duration of the activity or experience to match the sequence/intensity of the activity/experience as well as users biometrics, or visual or audio cues/inputs.

The wellbeing application 1010 or recommendation system 1100 can use different data processing techniques to generate the emotional signature. For example, the wellbeing application 1010 or the recommendation system 1100 can receive data sets (e.g. that can be extracted from aggregated data sources), extract metrics from the aggregated data sources, and generate the emotional signature for improved wellbeing using the extracted metrics.

In some embodiments, the wellbeing application 1010 can transmit the emotional signature to the recommendation system 1100 along with the recommendation request. In response, the wellbeing application 1010 updates its interface to display visual effects based on the emotional signature, and also based on the activity recommendation received by the recommendation system 1100. The wellbeing application 1010 can connect to the recommendation system 1100 to display the generated recommendation at the interface, or trigger other updates to the interface based on the recommendation (e.g. change an activity provided by the interface).

While in the above-described embodiment the processing of the user data, the determination of the emotional signatures, and the generation of the recommendations have been described as being performed by hardware servers 10, in other embodiments such steps may be performed by user device 16, provided that user device 16 has access to the required instructions, techniques, and processing power. Servers 10 can have access to greater processing power and resources than user devices 16, and therefore may be better suited to carrying out the relatively resource-intensive processing of user data obtained by user devices 16 and across channels.

In some embodiments, the recommendation system 1100 stores classifiers for generating data defining physical or behavioural characteristics of the user. The recommendation system 1100 can compute the activity metrics, cognitive-affective competency metrics, and social metrics using the classifiers and features extracted from multimodal feature extraction. The multimodal feature extraction can extract features from image data, video data, text data, and so on.

In some embodiments, the recommendation system 1100 stores user models corresponding to the users. The recommendation system 1100 can retrieve a user model corresponding to a user and computes the emotional signature of the user using the user model.

In some embodiments, the user device 16 connects to or integrates with an immersive hardware device that captures the audio data, the image data and the data defining the physical or behavioural characteristics of the user. The user device 16 can transmit the captured data to the recommendation system 1100 for processing. The user device 16 connects to the immersive hardware device using Bluetooth, or other communication protocol.

In some embodiments, the recommendation system 1100 stores a content repository and has a content curation engine that maps the activity recommendations to recommended content and transmits the recommended content to the interface of application 1010.

In some embodiments, the interface of application 1010 further comprises a voice interface for communicating activity recommendations for the user session received in response to the recommendation request. The voice interface can use speech/text processing, natural language understanding and natural language generation to communicate activity recommendations and capture user data.

In some embodiments, the interface of application 1010 access memory storing mood classifiers to capture the data defining physical or behavioural characteristics of the user.

In some embodiments, the recommendation system 1100 computes activity metrics, cognitive-affective competency metrics, and social metrics with classifiers using the user data for the user session and the user records and multimodal feature extraction that processes data from multiple modalities. The recommendation system 1100 uses multimodal feature extraction for extracting features and correlations across the image data, the data defining the physical or behavioural characteristics of the user, the audio data, and the text input. Multimodal signal processing analyzes user data through several types of measures, or modalities such as facial analysis; body analysis; eye tracking; behavioural analysis; social network or graph analysis; location analysis; user activity analysis; voice analysis; and text analysis, for example, and extracts features from the processed data.

In some embodiments, non-transitory memory stores classifiers for generating data defining physical or behavioural characteristics of the user, and the recommendation system 1100 computes the activity metrics, cognitive-affective competency metrics, and social metrics using the classifiers and features extracted from the multimodal feature extraction.

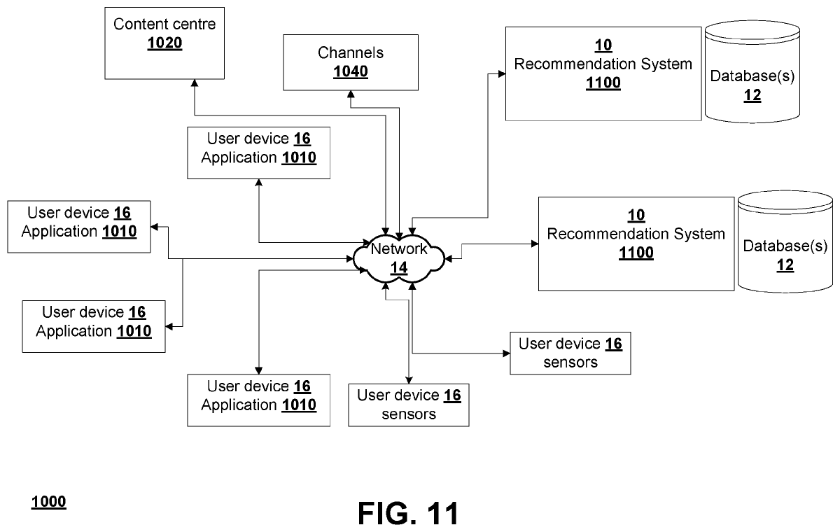

FIG. 11 illustrates another example of a wellbeing system 1000 with a wellbeing application 1010 that uses recommendation system 1100 to provide activity recommendations based on user data captured across the distributed system 1000. FIG. 11 is an example configuration with reference to components of FIG. 1 to illustrate that recommendation system 1100 can be referenced as a hardware server 10, for example. The wellbeing application 1010 can reside on a user device 16, for example.

The wellbeing application 1010 has an interface that receives a recommendation request and provides an activity recommendation in response to the request. The wellbeing application 1010 has the interface to provide the recommendations derived based on user data, activity metrics, and an emotional signature of a user. The wellbeing system 1000 can provide activity recommendations for different user sessions that can be defined by time periods. The wellbeing system 1000 can process user data based on the different user sessions defined by time periods. For example, wellbeing application 1010 can send a recommendation request to the recommendation system 1100 to start a user session for a time period. The user session maps to a user by a user identifier. The user session can define a set of captured user data (including captured real-time data), one or more emotional signatures, and one or more activity recommendations. A user session link a group of users in some examples. Each user session can have a recommendation request and a corresponding one or more activity recommendations. Each user session can be identified by the system 1000 using a session identifier stored in records of database 12. The recommendation request can indicate the session identifier, or the recommendation system 1100 can generate and assign as session identifier in response to receiving a recommendation request. The recommendation system 1100 or hardware server 10 and the interface of wellbeing application 1010 can exchange the session identifier via the API, for example. The recommendation system 1100 can store extracted metrics in association with a session identifier to map the data values to user sessions. The recommendation system 1100 can use data values from previous user sessions to compute emotional signatures and activity recommendations for a new user session. The previous user sessions can relate to the same user or different users.

As shown, the user devices 16 can have the interfaces of wellbeing applications 1010 to provide activity recommendations for the user sessions. The user devices 16 can also have sensors to capture (near) real-time user data during the time period of the user session (or proximate thereto) to determine the emotional signature of a user for the time period. A user session can be defined by one or more time periods or segments of a time period. A user session can map to one user identifier or multiple user identifiers.

The recommendation system 1100 or hardware server 10 receives input data from different data sources, such as content center 1020, user devices 16, and channels 1040 to compute different metrics for computation of the emotional signatures. The recommendation system 1100 or hardware server 10 computes the emotional signature for the user for the time period of the user session using the captured (near) real-time user data, along with other user data. The recommendation system 1100 can access records in databases 12, for example. The recommendation system 1100 can compute similarity measures across records for computation of the emotional signature of the user for the time period of the user session.

The recommendation request can relate to a time period of the user session and the activity recommendation generated in response to the request can relate to the same time period. The system 1000 can store the activity recommendation with the session identifier in records. In some embodiments, the wellbeing application 1010 can determine the activity recommendation or the emotional signature. In some embodiments, the wellbeing application 1010 can extract metrics from captured user data and transmit the extracted metrics to the recommendation system 1100 or hardware server 10. The wellbeing application 1010 has an interface that can display the activity recommendation at the user device 16 or otherwise provide the activity recommendation such as by audio or video data.

The example illustration shows multiple users devices 16 with wellbeing applications 1010 and multiple user devices 16 with sensors. The user devices 16 can connect to recommendation system 1100 or hardware server 10 to exchange data for user sessions. The recommendation system 1100 or hardware server 10 can aggregate or pool data from the multiple users devices 16 and send activity recommendations to interfaces of wellbeing applications 1010. The recommendation system 1100 can coordinate timing of the real-time data collection from a group of users corresponding to a set of user devices 16 and can coordinate timing and content of activity recommendations for the interfaces of the wellbeing applications 1010 for each user of the group of users. A group of users can be assigned to a user session, for example, to coordinate data and messages. For example, recommendation system 1100 can generate the same activity recommendation for transmission to wellbeing applications 1010 for each user of the group of users of the user session. The wellbeing application 1010 can be linked to a user by a user identifier that can be provided as credentials at the interface or generated using data retrieved by the interface from the user device 16. The user identifier can map to a user record in the database 12. The session identifier can also map to one or more user identifiers in the database 12. During a registration process, the interface of the wellbeing application 1010 can exchange the user identifier with the recommendation system 1100 or hardware server 10 via the API, for example.

The example illustration shows recommendation system 1100 or hardware server 10 exchanging data between multiple users devices 16 with wellbeing applications 1010 and multiple user devices 16 with sensors. The recommendation system 1100 or hardware server 10 can have increased computing power to efficiently compute data values from the aggregated user data. Each user device 16 does not have to store the aggregated user data and does not have to process similarity measures across a group of users. Each user device 16 does not have to exchange data with all the user devices 16 in order to access the benefits of data aggregation. Instead, the user device 16 can exchange data with the recommendation system 1100. The recommendation system 1100 or hardware server 10 can store the aggregated user data and process similarity measures across a group of users and exchange data with the user device 16 based on the results of its computations. The user device 16 can capture real-time user data during user sessions for the recommendation system 1100 or hardware server 10, or can perform computations for the user session using the real-time user data and data received from the recommendation system 1100. The wellbeing applications 1010 can extract metrics from captured user data and transmits the extracted metrics to the recommendation system 1100. The wellbeing applications 1010 can exchange data and commands with the recommendation system 1100 during user sessions using the API. The extracted metrics can correspond to parameters for the API, as an example. The wellbeing applications 1010 can transmit extracted metrics to the recommendation system 1100 using the API. The wellbeing applications 1010 can extract metrics from captured user data so that the metrics might not reveal all sensitive user data. In some embodiments, the wellbeing applications 1010 can transmit the metrics to the recommendation system 1100 using the API instead of all the sensitive user data.

The recommendation system 1100 or hardware server 10 can serve a large number of wellbeing applications 1010 to scale the system 1000 to collect a corresponding large amount of data for the computations. The system 100 can have multiple recommendation systems 1100 or hardware servers 10 to serve sets of user devices 16, for example, and provide increased processing power and data redundancy.

The recommendation system 1100 or hardware server 10 can receive user data relating to a user for user sessions from a plurality of channels 1040. The user data involves different types of data such as image data relating to the user, text input relating to the user, data defining physical or behavioural characteristics of the user, and audio data relating to the user;

The recommendation system 1100 or hardware server 10 can implement pre-processing steps on the raw data received from different channels 1040. Examples include importing data libraries; data cleaning or checking for missing values/data; smoothing or removing noisy data and outliers; data integration; data transformation; and normalization and aggregation of data.

The wellbeing applications 1010 can exchange data and commands with the recommendation system 1100 using the API, such as metrics extracted from the captured user data. The wellbeing application 1010 or the recommendation system 1100 can generate activity metrics, cognitive-affective competency metrics, and social metrics by processing the user data using one or more hardware processors configured to process the user data from the plurality of channels 1040. This includes captured user data for a time period given that the activity recommendation corresponds to the time period. The captured user data for the time period is used to compute an emotional signature for the user for the time period.

The activity metrics, cognitive-affective competency metrics, and social metrics define “physical” metrics and “cognitive” metrics for the system 1000. Raw data is ingested by the system 100 from the different channels 1040 and mapped to these definitions of “physical” metrics and “cognitive” metrics by the system 100. The metrics can have corresponding values based on the processed user data. The system 1000 abstracts from the raw user data using the “physical” metrics and “cognitive” metrics to provide an improved way to compute values for an emotional signature for the user for the time period.

For example, the system 1000 measures the physiological condition of the user using sensors (accelerometer, heart rate monitor, breath rate monitor) to capture real-time user data and processes user data to measure physiological conditions (e.g. measuring heart rate, heart rate variability) by assigning values to different metrics. The system 1000 can define a ‘physical’ metric or a fluidity score during a workout activity that can be computed by user data captured using physiological sensors of user device 16 with or without a camera, for example. The system 1000 can define a connectedness metric using heart rate and heart rate variability during a workout activity, as another example.

For example, the system 1000 measures cognitive metrics using definitions based on text inputs (with predefined answers and free text answers with predefined features extracted to predefined questions), daily behaviour (e.g. extracted from user's device 16 like app usage, music consumption, number of outgoing calls), voice (power spectrum of the speech signal can correlate emotions like neutral, anger, joy, sadness), body language extracted from image data (posture, special location and orientation of joints like wrist and hands can correlate emotions like happy, sad, surprise, fear, anger, disgust, neutral); eye movement (saccade duration, fixation duration, pupil diameter can correlate to positive, neutral or negative emotional state). Another example is brain activity data (e.g. N400 response).

As another example, the system 1000 can measure cognitive metrics using higher level state definitions such as intention/awareness, attention, motivation, emotion regulation, perspective-taking/insight, self-compassion and compassion towards others.

The system 1000 can measure physical metrics and cognitive metrics from captured user data for a user session and then compute the emotion signature for the user session using the physical metrics and cognitive metrics. The system 1000 can measure physical metrics and cognitive metrics from text base interaction, free text response and extracts features from the free responses for computing to the emotional signature. A user can be in front of mirror device with a camera to capture images of gestures and audio data of speech for the user session which can be used to compute additional metrics such as tone or body posture.

The system 1000 can measure physical metrics as state metrics, such as being “happy” can be smile detected in image data or posture or tone from audio data. The system 1000 can measure trait metrics or personality more constant features. For example, to measure the level of attention or focus a user has at a given time the interface can prompt a predefined question: ‘how focused are you feeling right now?’ with a 1-7 Likert scale response. The interface ask a specific or general question and extract any features related to feeling focused through a free text response. The system 1000 can also consider communication messages between users, such as text conversation data between two users and extract features related to a user describing feeling focused. The system 1000 can also consider reaction times to digital interactions on the phone or other devices (e.g. button clicks). The system 1000 can also consider device usage data to measure how much time a user was on task or focused, or off-task and not focused in the day. We could use visual eye tracking to measure attention and focus to a particular task.

The wellbeing application 1010 or recommendation system 1100 can extract metrics from image data and the data defining the physical or behavioural characteristics of the user, using at least one of: facial analysis; body analysis; eye tracking; behavioural analysis; social network or graph analysis; location analysis; user activity analysis. Examples of different facial feature extraction techniques and image processing techniques include observation techniques such as those based on the Facial Action Coding System where observable activity of specific muscle group are labeled and coded as action units by human coders, record muscles activities with facial electromyography; facial expression coding system (FACES) developed by Berkeley (https://esilab.berkeley.edu/wp-content/uploads/2017/12/Kring-Sloan-2007.pdf) the entire contents of which is hereby incorporated by reference.

The wellbeing application 1010 or recommendation system 1100 can extract metrics from the audio data, using different voice processing techniques. For example, metrics can be values based on non-linguistic verbal interactions for emotion states (e.g. laughter, sighs)

The wellbeing application 1010 or recommendation system 1100 can extract metrics from the text input using text analysis and different natural language understanding techniques to extract features from text data, including meaning and sentiment analysis.

The wellbeing application 1010 or recommendation system 1100 can compute activity metrics, cognitive-affective competency metrics, and social metrics. The wellbeing application 1010 or recommendation system 1100 can determine, based on the cognitive-affective competency metrics and social metrics generated from the processed user data, one or more states of one or more cognitive-affective competencies of the user. Examples of state classification happy, sad, disgust, moment of insight, giving, compassion, compelled to help, jealousy, energized, being focus, surprise, fear, anger, curious, aware, unaware. The wellbeing application 1010 or recommendation system 1100 can define multiple states and select states for user sessions or time periods. The definitions for states can relate to ‘readiness to grow’ as another example.

The wellbeing application 1010 or recommendation system 1100 can compute an emotional signature of the user for the user session based on the one or more states of the one or more cognitive-affective competencies of the user. The system 1000 can map states to the emotion signature values or parameters. By way of example for physical fitness, training over years contributes to a general fitness level which does not change quickly. If a user has done hard training recently, the day after the training session the user might be really tired and so their readiness to train might be low. The system 1000 can consider metrics for a user computed based on data captured before or prior to the time period of the user session, along with metrics for the user computed based on data captured during the time period of the user session. The system 1000 can use a weighting or ratio for the metrics to compute the emotional signature or additional metrics for the session. The emotion signature can be computed using metrics for different dimensions of emotion, such as Awareness, Regulation, Compassion (ARC) dimensions of emotion. Within each dimension there are different states that could be detected by the system 1000 that would be attributed to that dimension. For awareness, the system 1000 can define subdimensions like reflectiveness, mindfulness and purposefulness. The interface can display an initial questionnaire to receive input data for a user session to measure as a trait level metric. However, with different real time data inputs the system 1000 can measure discrete states at different time intervals (using data corresponding to the different time intervals) over a time period or across different user sessions. For example, a user would be in a state of reflectiveness when they are labeling a current or past experience and expressing things like emotions or feelings they had during that experience in words either spoken or written. To detect this state, a person's spoken language or written language could be processed and features extracted that relate to the expression of emotions in relation to an event.

The wellbeing application 1010 or recommendation system 1100 can define emotion signatures as functions or sets of values. An emotion signature definition can model ARC dimensions and consider values for metrics for ARC dimensions as profiles of values (metric 1, metric 2, metric 3) with different versions or combinations depending on the values that can be assigned. An example is profile of values is (A, R, C) where each value can be high or low, with different versions of profiles such as: (high-high-high) (high-high-low) (high-low-high) (high-low-low) (low-low-high) (low-low-low) (low-high-high) (low-low-high). The different versions of profiles can map to different emotional signatures. The profiles can be stored in records of database 12, for example.

The wellbeing application 1010 or recommendation system 1100 can select an emotional signature from a group of emotional signatures using confidence scores or distribution rules, for example. As an example, the rules can correspond to default to the population distribution work or the profile that best represents most users, e.g. lower on self-compassion. The interface can also prompt for more information to capture additional user data (e.g. digital or virtual coach or conversational agent style interface) to select an emotional signature from the group of emotional signatures.

The recommendation system 1100 can automatically generate, based on the emotional signature of the user and the activity metrics, one or more activity recommendations for the interface. The recommendation system 1100 transmits the activity recommendations to the interface in response to a recommendation request. Recommendations can be based on thresholds of scores from predefined questions and Likert scale responses. Recommendations can be based on advanced data points and complex data collection and analysis methods.

The wellbeing application 1010 can provide a client interface for an automated coaching application to provide automated activity recommendations for user sessions using physical and cognitive metrics extracted from captured user data.

The wellbeing application 1010 can be a mobile companion application (residing an a computer device) for a separate hardware device that captures user data. The separate hardware device can also have an interface that can deliver recommendations in coordination with the wellbeing application 1010. Within the companion application, the wellbeing application 1010 has a conversational agent interface to offer activity or content recommendations. The system 1000 can have a combination of a hardware device with sensors to capturing user data and a companion mobile wellbeing application 1010 on a separate hardware device to exchange data with the recommendation system 1100. A hardware device with the companion mobile wellbeing application 1010 can trigger a digital coaching session to recommend different styles and types of mental training activities (e.g. concentrative meditation, open-monitoring meditation, compassion meditation), physical activities (yoga, walking, spin etc.), peer coaching activities (e.g. discussions on various topics of emotional development, mirroring or eye gazing, practicing listening to a partner without speaking), and so on.

The recommendation system 1100 can implement a state-based personality measure for the emotion signature. State-based personality is a measurement that changes over a period of time based on collected data. Initially, recommendation system 1100 can collect a brief trait personality measure. Then over time, through the collection of states, recommendation system 1100 can dynamically re-compute the emotion signature over the period of the time (e.g. at intervals, at detected events) of the user session so that it would be dynamically be changing based on the states over time during each user session. The recommendation system 1100 can use a rolling average based on the states measured, for example.

The wellbeing application 1010 can implement natural language generation techniques for communicating the activity recommendations or output received from the recommendation system 1100. The wellbeing application 1010 can used advanced data points and user preferences, various types of psychographic and demographic data, transaction data on products linked to various healthy activities (running, yoga, etc.), and other contextual information on life goals and values. The wellbeing application 1010 can use this data to further contextualize the output received from the recommendation system 1100 to develop of tailored interface experience for the user.

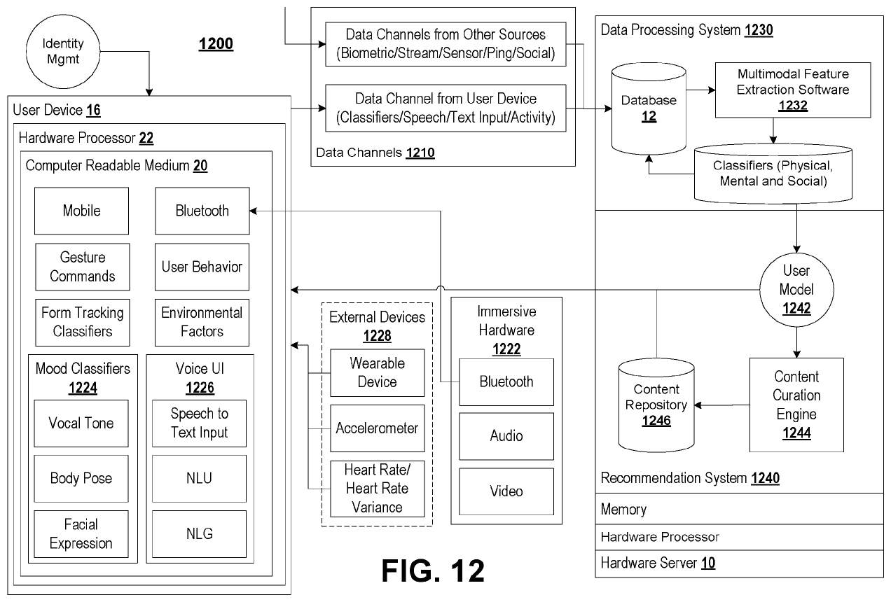

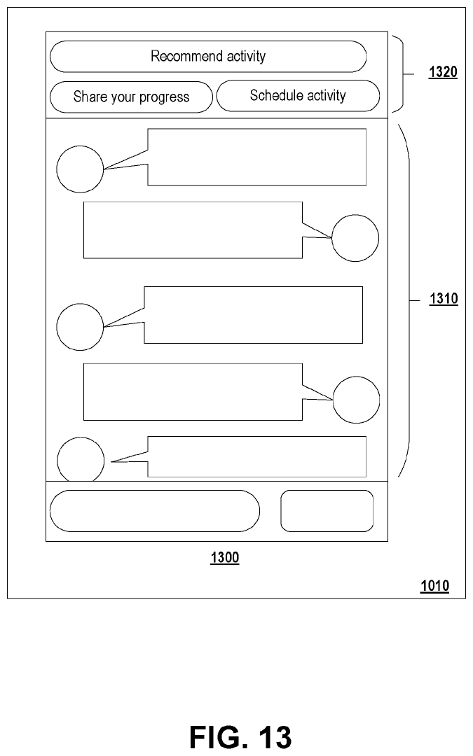

FIG. 12 illustrates an example of a wellness system 1200 that provides activity recommendations based on user data. FIG. 12 is an example configuration with reference to components of FIG. 1 to illustrate that recommendation system 1200 can be referenced as a hardware server 10, for example.

The wellness application 1200 collects and aggregates user data from a plurality of channels 1210. The plurality of channels 1210 provide data to a server 10. In some embodiments, the data is received at server 10 and processed by a data processing system 1230 and is used to create a user model 1242. A recommendation system 1240 uses the user model to provide recommendations. Content is delivered to a user device 16 based on the recommendations. The user device 16 is configured to collect user data and provide it to the wellness system through one or more of the plurality of data channels.

The user device 16 has non-transitory computer readable medium 20 storing mobile data, The user device 16 has non-transitory computer readable medium 20 storing different programs to configure the hardware processor 22. The user device 16 has a Bluetooth in this example for communication with other components of system 1200. The user device 16 provides one or more different forms of user data and device related data (including mobile data). The user data can include image data relating to the user, text input relating to the user, data defining physical or behaviour characteristics of the user, and audio data relating to the user. The user device may collect the user data through one or more of gesture commands, user behaviour, environmental factors, form tracking classifiers, mood classifiers, voice UI, or user data provided by external devices. In some embodiments, the user device 16 has one or more mood classifiers 1224 that collect data from one or more of the user's vocal tone, body pose, or facial expression. In some embodiments, the mood classifiers 1224 can compute cognitive-affective competency metrics based on data stored on or accessible to user device 16. In some embodiments, the mood classifiers 1224 can compute behavioural characteristics of the user based on image data stored on or accessible to user device 16. In some embodiments, the user device 16 has a voice UI 1226 that has speech to text input, natural language understanding, and natural language generation. The voice UI 1126 can be a conversational agent, for example. In some embodiments, the mood classifiers 1224 can be connected to user models 1242. The user device 16 and the system 1230 can exchange data between the mood classifiers 1224 and the user models 1242. In some embodiments, each user device 16 (or associated user) has a corresponding user model 1242 to compute data for the specific user device 16.

The external device 1228 or immersive hardware 1222 can transmits user data collected by its sensors to user device 16 for processing. The user device 16 can implement processing operations on the collected data from external device 1228 or immersive hardware 1222. The user device 16 can interact with system 1230 to implement processing operations on the collected data from external device 1228.

In some embodiments, the user device 16 collects user data from one or more external devices 1228. For example, in some embodiments, the user device 16 collects user data from an external device 1228 with one or more wearable devices, accelerometers, heart rate sensors, and heart rate variability sensors. The one or more external devices 1228 may be physically or communicatively coupled to the user device 16 to exchange data.

In some embodiments, the user device 16 collects user data from one or more immersive hardware devices 1222. The one or more immersive hardware devices are physically or communicatively coupled to the user device 16. In some embodiments, the immersive hardware devices 1222 are coupled to the user device 16 using Bluetooth. In some embodiments, the immersive hardware devices 1222 collect one or more of audio data relating to the user and video image data relating to the user. In some embodiments, the immersive hardware device 1222 can display audio or visual content. In some embodiments, the immersive hardware device 1222 can provide data as part of the immersive channels 1040 shown in FIG. 8.

In an example embodiment, the user device 16 and the immersive hardware device 1222 can provide a real-time interactive coaching session. The immersive hardware device 1222 can be use to collect data and communicate data related to activity recommendations and recommended content. The interactive coaching session can involve one or more activity recommendations, for example. The interactive coaching session can involve a sequence of activity recommendations. The sequence of activity recommendations can vary depending on a corresponding user and also based on feedback from previous sessions or previous activity recommendations in the sequence of activity recommendations for the coaching session. The user device can have an application or software program installed thereon (e.g. application 1010) that can exchange data with system 1230 to install trained user models 1242 on the device 16 as mood classifiers 1224 or as other trained models for user behaviour. The user device 16 and the immersive hardware device 1222 can exchange data for real-time interaction. The immersive hardware device 1222 can have an interface that can update based on the real-time interaction. The immersive hardware device 1222 can transmit data to the user device 16 which can run trained models on the data. The user device 16 can transmit output data from the trained models to the immersive hardware device 1222. The user device 16 can have increased processing resources for processing collected data (e.g. hardware processor 22) than immersive hardware device 1222 in some example embodiments. The immersive hardware device 1222 can also have a hardware processor in some embodiments.

The user device 16 receives activity recommendations which can be referred to as content recommendations in some example embodiments. The activity can be associated with data or content defined in the content recommendations. For example, the activity can be an exercise and the content can be used as part of the exercise. The voice UI 1226 can communicate the content recommendations (or audio files or text data therein) during the activity or as the activity recommendation, for example. The content recommendations can also include audio or video files. The content recommendation system 1240 can generate content recommendations and transmit the content recommendations to the user device 16. The user device 16 receives recommended content provided by the content recommendation system 1240. In some embodiments, the user device 16 has an interface that displays this content to the user. In some embodiments, the user device 16 transmits the content to an immersive hardware device 1222 which displays the content or otherwise communicates the content. In some embodiments, the user device 16 uses the voice UI 1226 with a conversational agent to deliver the recommended content to the user.

The data collected by the user device 16 directly or through other devices (e.g. immersive hardware device 1222) communicatively coupled to the user device 16 can be transferred or transmitted to the server 10 through data channels 1210. The server 10 may also receive data from one or more data channels 1210 from sources other than the user device 16.

The user data is stored in user records in a database 12 contained in the memory of the server 10. The user data is processed by a data processing system 1230 having a multimodal feature extraction software 1232. The data processing system 1230 can process the user data by extracting features from the user data using the multimodal feature extraction software 1232 and process the extracted features using different classifiers. The classifiers can relate to physical, mental, and social classification models, for example. The output of the classifiers can be stored in database 12. The classifiers (physical, mental, and social) can interact with user models 1242 compute cognitive-affective competency metrics, states of cognitive-affective competencies, and emotional signatures for different users. The user models 1242 can have a user model 1242 corresponding to a specific user. The user model 1242 corresponding to a specific user can update over time as additional user data is collected by the data processing system 1230. A user model 1242 can also map to categories or types of specific users. The user model 1242 corresponding to a specific user type can update over time as user data of the user type is collected by the data processing system 1230. The user type can define a set of users and the user data used to update the user model 1242 corresponding to a specific user type can correspond to data from the set of users.

The data processing system 1230 can generate different metrics using the extracted features. The data processing system 1230 computes activity metrics, cognitive-affective competency metrics, and social metrics. To calculate these metrics, the data processing system 1230 uses at least one of facial analysis, body analysis, eye tracking, behavioural analysis, social network or graph analysis, and user activity analysis. For audio data, the data processing system 1230 uses voice analysis. For text input, the data processing system 1230 uses text analysis. In some embodiments, the multimodal feature extraction software 1232 can use facial analysis, body analysis, eye tracking, behavioural analysis, social network or graph analysis, and user activity analysis to extract features from the user data. The data processing system 1230 can use the multimodal feature extraction software 1232 to extract features and generate metrics. The activity metrics, cognitive-affective competency metrics, and social metrics are stored in the memory of the server 10.

The cognitive-affective competency metrics and social metrics are used by system 1200 to compute one or more states of one or more cognitive-affective competencies. The one or more cognitive-affective competencies are stored in the database 12 as part of the user records.

The data processing system 1230 computes an emotional signature of the user for the user session using at least some user data collected over the time period based on one or more states of the one or more cognitive-affective competencies of the user and using the emotional signature records. The data processing system 1230 can re-compute the emotional signature of the user for the user session over the time period.

In some embodiments, the recommendation system 1240 generates and updates user models 1242 for processing data to compute the emotional signature of the user, the activity metrics, the activity recommendation records, and the user records. The classifiers can interact with the user models 1242 to compute metrics.

In some embodiments, the user model 1242 is used to generate activity recommendations and the user model 1242 can correspond to different emotional signatures. In such an embodiment, the classifiers can be used to compute metrics and the emotional signature for the user session. The computed emotional signature can be used to identify a user model 1242 to compute the activity recommendations. A user can be associated with different user models 1242, each corresponding to different emotional signatures. In some embodiments, multiple users can be associated with a user model 1242 that corresponds to different emotional signatures. The activity recommendations are then used to retrieve content from a content repository 1246. The content is then delivered to the user device 16. The content repository 1246 can define different content for different activity recommendations. The content repository 1246 can retrieve a first set of content for a first activity recommendation and a second set of content for a second activity recommendation, for example.

In some embodiments, one or more of the computations of the data processing system 1230 and/or the recommendation system are performed by one or more of the user device 16, one or more immersive hardware devices 1222, and one or more external devices 1228. The computations can be distributed across different devices based on available resources in order to improve processing efficiency across the wellness system 1200 and to address communication or network constraints.

The user device 16 can install or interact with a software program for identity management to authenticate a user device 16 or otherwise associate the user device 16 with an identifier. The user device 16 can also store wellness application 1010 that can involve different components shown, such as mood classifiers 1224 and voice UI 1226. That is, wellness application 1010 can have a voice UI 1226, for example.

FIG. 13 illustrates an example of a user interface 1300 of a wellness application 1010. The user interface 1300 displays instant messaging conversations between a first user and a second user 1310. The second user 1310 can be a virtual coach and the message can be generated automatically by the system 1200 or based on input from one or more coaches. The user interface 1300 also has selectable indicia 1320 to trigger a recommendation request to update the user interface 1300 with one or more activity recommendations. Upon selection of the selectable indicia 1320, the wellness application 1010 can transmit a recommendation request to recommendation system 1240, for example. In response, the wellness application 1010 receives activity recommendations or associated recommended content, and updates the user interface 1300 to display or communicate the activity recommendations or associated recommended content. The activity recommendations may include content that can be provided to the user as a message shown on interface 1300. The messaging conversation can also request additional input data from user device 16 before generating the activity recommendations. For example, activity recommendations and messages may include recommending a workout to the second user, sharing the first user's progress with the second user, and scheduling a workout with the second user. If the first user selects a particular activity, the wellness application 1010 can perform actions corresponding to the selection. For example, if the first user selects an activity recommendation to schedule a workout with the second user, the wellness application 1010 may present an update interface 1300 to schedule a time for the workout and send an invitation to the second user. The activity recommendations are generated automatically by the recommendation system 1320 of FIG. 13, for example. The wellness application 1010 can be stored on non-transitory computer readable medium and is executable by a hardware processor to implement operations described herein.

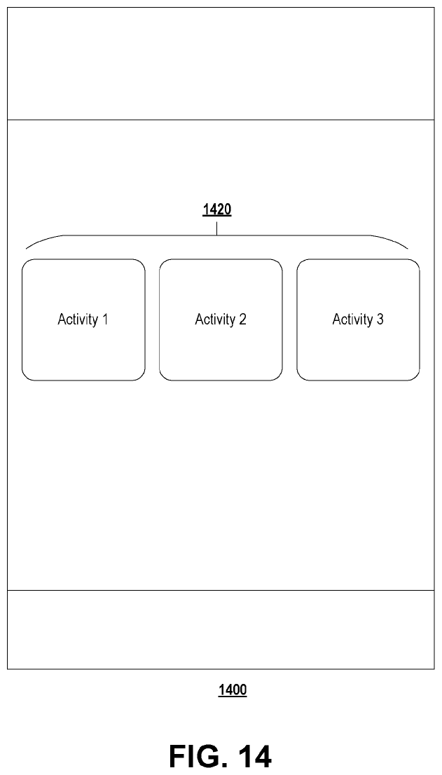

FIG. 14 illustrates an example of another user interface 1400 of a wellness application 1010. The user interface 1400 displays a plurality of activity recommendations 1420 which the user may select. For example, the activity recommendations may include a recommended exercise class, and when the user selects the exercise class, the wellness application may cause the user to add the user to the exercise class. The activity recommendations are generated automatically by the recommendation system 1320. Upon selection of one of the activity recommendations 1420, the wellness application 1010 can transmit data corresponding to the selected activity recommendation to recommendation system 1320 or store the data corresponding to the selected activity recommendation in memory as part of user record, for example. The selected activity recommendation can be used as user data for generating additional activity recommendations for subsequent user sessions or for users with similar emotional signatures, for example.

The word “a” or “an” when used in conjunction with the term “comprising” or “including” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

The terms “coupled”, “coupling” or “connected” as used herein can have several different meanings depending on the context in which these terms are used. For example, the terms coupled, coupling, or connected can have a mechanical or electrical connotation. For example, as used herein, the terms coupled, coupling, or connected can indicate that two elements or devices are directly connected to one another or connected to one another through one or more intermediate elements or devices via an electrical element, electrical signal or a mechanical element depending on the particular context. The term “and/or” herein when used in association with a list of items means any one or more of the items comprising that list.

As used herein, a reference to “about” or “approximately” a number or to being “substantially” equal to a number means being within +1-10% of that number.

While the disclosure has been described in connection with specific embodiments, it is to be understood that the disclosure is not limited to these embodiments, and that alterations, modifications, and variations of these embodiments may be carried out by the skilled person without departing from the scope of the disclosure.

It is furthermore contemplated that any part of any aspect or embodiment discussed in this specification can be implemented or combined with any part of any other aspect or embodiment discussed in this specification.

1. A system for monitoring a user over a user session using one or more sensors and providing an interface with activity recommendations for the user session, the system comprising:
non-transitory memory storing activity recommendation records, emotional signature records, and user records storing user data received from a plurality of channels, wherein the user data comprises image data relating to the user, text input relating to the user, data defining physical or behavioural characteristics of the user, and audio data relating to the user;
a hardware processor programmed with executable instructions for an interface for obtaining user data for a user session over a time period, transmitting a recommendation request for the user session, and providing activity recommendations for the user session received in response to the recommendation request;
a hardware server coupled to the memory to access the activity recommendation records, the emotional signature records, and the user records, the hardware server programmed with executable instructions to transmit the activity recommendations to the interface over a network in response to receiving the recommendation request from the interface by:
extracting physical metrics of the user and cognitive metrics of the user from the user data and computing activity metrics, cognitive-affective competency metrics, and social metrics using the user data for the user session and the user records and multimodal feature extraction that:
for the image data and the data defining the physical or behavioural characteristics of the user, implements at least one of: facial analysis; body analysis; eye tracking; behavioural analysis; social network or graph analysis; location analysis; user activity analysis;
for the audio data, implements voice analysis; and
for the text input implements text analysis;
computing one or more states of one or more cognitive-affective competencies of the user based on the cognitive-affective competency metrics and the social metrics;
computing an emotional signature of the user at time intervals during the time period of the user session based on the one or more states of the one or more cognitive-affective competencies of the user using the physical metrics of the user and the cognitive metrics of the user, and the emotional signature records;
monitoring the emotional signature of the user over the time intervals during the time period; and
computing the activity recommendations based on the emotional signature of the user, the activity metrics, the activity recommendation records, and the user records; and
a user device comprising one or more sensors for capturing user data during the time period, and a transmitter for transmitting the captured user data to the interface of the hardware processor or the hardware server over the network to compute the activity recommendations.

2. The system of claim 1 wherein the non-transitory memory stores classifiers for generating data defining physical or behavioural characteristics of the user, and the hardware server computes the activity metrics, cognitive-affective competency metrics, and social metrics using the classifiers and features extracted from the multimodal feature extraction.

3. The system of claim 1 wherein the non-transitory memory stores a user model corresponding to the user and the hardware server computes the emotional signature of the user using the user model.

4. The system of claim 1 wherein the user device connects to or integrates with an immersive hardware device that captures the audio data, the image data and the data defining the physical or behavioural characteristics of the user.

5. The system of claim 1 wherein the non-transitory memory has a content repository and the hardware server has a content curation engine that maps the activity recommendations to recommended content and transmits the recommended content to the interface.

6. The system of claim 1 wherein the hardware processor programmed with executable instructions for the interface further comprises voice interface for communicating activity recommendations for the user session received in response to the recommendation request.

7. (canceled)

8. The system of claim 1 further comprising one or more modulators in communication with one or more ambient fixtures to change to change external sensory environment based on the activity recommendations, the one or more modulators being in communication with the hardware server to automatically modulate the external sensory environment of the user during the user session.

9. The system of claim 8, wherein the one or more ambient fixtures comprise at least one of a lightening fixture, an audio system, an aroma diffuser, a temperature regulating system.

10. The system of claim 1 further comprising a plurality of user devices, each having different types of sensors for capturing different types of user data during the user session, each of the plurality of devices transmitting the captured different types of user data to the hardware server over the network to compute the activity recommendations.

11. The system of claim 1 further comprising a plurality of hardware processors for a group of users, each hardware processor programmed with executable instructions for a corresponding interface for obtaining user data for a corresponding user of the group of users for the user session over the time period, and providing activity recommendations for the user session received in response to the recommendation request, wherein the hardware server transmits the activity recommendations to the corresponding interfaces of the plurality of hardware processors in response to receiving the recommendation request from the corresponding interfaces and computes the activity recommendations for the group of users.

12. (canceled)

13. The system of claim 1, wherein the interface can receive feedback on the activity recommendations for the user session, transmit the feedback to the hardware server, wherein the interface can transmit another recommendation request for the user session, and provide additional activity recommendations for the user session received in response to the other recommendation request.

14. (canceled)

15. The system of claim 1, wherein the interface obtains additional user data after providing the activity recommendations for the user session, the additional user data captured during performance of the activity recommendations by the user.

16. The system of claim 1, wherein the interface transmits another recommendation request for another user session, and provides updated activity recommendations for the other user session received in response to the other recommendation request, the updated activity recommendations being different that the activity recommendations.

17. The system of claim 1, wherein the one or more activity recommendations comprise content recommendations, wherein the activity is associated with content defined in the content recommendations for display or playback on the hardware processor, wherein the activity is exercise and the content is used as part of the exercise.

18. The system of claim 1, wherein the interface is a coaching application and the one or more recommended activity is delivered by a matching coach.

19. The system of claim 1, wherein the activity recommendations are selected from the group consisting of: pre-determined classes selected from a set of classes stored in the activity recommendation records, and a program with variety of content for the interface to guide user's interactions or experience for a prolonged time.

20. (canceled)

21. A computer-implemented method comprising:
receiving user data relating to a user from a plurality of channels at a hardware server and storing the user data as user records in non-transitory memory, wherein the user data comprises image data relating to the user, text input relating to the user, data defining physical or behavioural characteristics of the user, and audio data relating to the user;
extracting physical metrics of the user and cognitive metrics of the user from the user data and generating activity metrics, cognitive-affective competency metrics, and social metrics by processing the user data using one or more hardware processors configured to process the user data from the plurality of channels by:
for the image data and the data defining the physical or behavioural characteristics of the user, using at least one of: facial analysis; body analysis; eye tracking; behavioural analysis; social network or graph analysis; location analysis; user activity analysis;
for the audio data, using voice analysis; and
for the text input using text analysis;
determining, based on the cognitive-affective competency metrics and social metrics generated from the processed user data, one or more states of one or more cognitive-affective competencies of the user;
determining an emotional signature of the user at time intervals during the time period of the user session based on the one or more states of the one or more cognitive-affective competencies of the user using the physical metrics of the user and the cognitive metrics of the user;
monitoring the emotional signature of the user over the time intervals during the time period;
automatically generating, based on the emotional signature of the user and the activity metrics, one or more activity recommendations for a user session;
transmitting the activity recommendations to a user interface at a hardware processor in response to a recommendation request;
updating the user interface at the hardware processor to provide the activity recommendations based on user preferences; and
modulating an external sensory actuators of an external sensory environment during the recommended activity in response to the hardware server or interface.

22. (canceled)

23. (canceled)

24. (canceled)

25. (canceled)

26. (canceled)

27. (canceled)

28. The method of claim 17, further comprising:
receiving user data relating to one or more additional users, wherein the user data comprises at least one of image data relating to the one or more additional users, text input relating to the one or more additional users, biometric data relating to the one or more additional users, and audio data relating to the one or more additional users;
processing the user data using at least one of: facial analysis; body analysis; eye tracking; voice analysis; behavioural analysis; social network analysis; location analysis; user activity analysis; and text analysis;
determining, based on the processed user data, one or more states of one or more cognitive-affective competencies of the one or more additional users;
determining an emotional signature of each of the one or more additional users;
determining users with similar emotional signatures;
predicting connectedness between users with similar emotional signatures; and
generating one or more activity recommendations for transmission to interfaces of users with similar emotional signatures.

29. The method of claim 17, further comprising:
determining, based on the processed user data, a personality type of the user,
wherein determining the emotional signature of the user is further based on the personality type of the user,
wherein the processed user data comprises personality type data, and wherein determining the personality type of the user comprises:
comparing the personality type data to stored personality type data indicative of correlations between personality types and personality type data.

30. (canceled)

31. (canceled)

32. The method of claim 17, further comprising:
determining, based on the processed user data, at least one of: one or more mood states of the user, one or more attentional states of the user, one or more prosociality states of the user, one or more motivational states of the user, one or more reappraisal states of the user, and one or more insight states of the user, and
wherein determining the one or more states of the one or more cognitive-affective competencies of the user is further based on the at least one of: the one or more mood states of the user, the one or more attentional states of the user, the one or more prosociality states of the user, the one or more motivational states of the user, the one or more reappraisal states of the user, and the one or more insight states of the user.

33. (canceled)

34. (canceled)

35. (canceled)

36. (canceled)

37. (canceled)

38. (canceled)

39. (canceled)

40. (canceled)
